SEQUENCE LISTING (53 sequences)

SEQ ID NO: 1 - DNA, 24 bp, Artificial Sequence
  5′ biotinylated sense primer designed from exon 3
catggtactgggagaacctgctca

SEQ ID NO: 2 - DNA, 27 bp, Mus Sp.
cccttctgtcgtcttctcgcagccgta

SEQ ID NO: 3 - DNA, 27 bp, Artificial Sequence
  sense primer used in first round of PCR derived from the adapt primer
tacggctgcgagaagacgacagaaggg

SEQ ID NO: 4 - DNA, 24 bp, Mus Sp.
ccggcatgatgaaggcgaagatga

SEQ ID NO: 5 - DNA, 23 bp, Mus Sp.
cggaaatccagggccttgaccgg

SEQ ID NO: 6 - DNA, 22 bp, Artificial Sequence
  sense primer designed from exon 11
cctccaggctcatttcagagag

SEQ ID NO: 7 - DNA, 48 bp, Artificial Sequence
  sense primer designed from exon 11
ggcgcgggatctgggccgatgatggaagctttctctaagtctgcattc

SEQ ID NO: 8 - DNA, 24 bp, Mus Sp.
ccaggaaaccagagcctcccacaa

SEQ ID NO: 9 - DNA, 22 bp, Mus Sp.
ggcgtgggacccagttagggca

SEQ ID NO: 10 - DNA, 24 bp, Mus Sp.
gaaaggcatcttccctctcgctgt

SEQ ID NO: 11 - DNA, 24 bp, Mus Sp.
ccacactgctcaccagctcatccc

SEQ ID NO: 12 - DNA, 24 bp, Mus Sp.
gtggaaccagagaagagcggcagg

SEQ ID NO: 13 - DNA, 24 bp, Mus Sp.
ctttgtggtggtcgaccagttgcc

SEQ ID NO: 14 - DNA, 24 bp, Mus Sp
ggacgctcagacgttccattctgc

SEQ ID NO: 15 - DNA, 1440 bp, Mus Sp.
ggcgcgggatctgggccgatgatggaagctttctctaagtctgcattccaaaagctcagacagagagatggaaatcaagaggggaagagttacctcagggcttgtccttgtaagaaactgacggagcctagggcagctgtgagaggaagaggctggggcgcctggaacccgaacactcttgagtgctctcagttacagcctaccgagtccgcagcaagcattcagaaccatggacagcagcgccggcccagggaacatcagcgactgctctgaccccttagctcctgcaagttgctccccagcacctggctcctggctcaacttgtcccacgttgatggcaaccagtccgacccatgcggtcctaaccgcacggggcttggcgggagccacagcctgtgccctcagaccggcagcccttccatggtcacagccatcaccatcatggccctctattctatcgtgtgtgtagtgggcctctttggaaacttcctggtcatgtatgtgattgtaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcacgttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagctagaaaatctggaagcagaaactgctccattgccctaactgggtcccacgcc

SEQ ID NO: 16 - DNA, 1614 bp, Mus Sp.
ggcgcgggatctgggccgatgatggaagctttctctaagtctgcattccaaaagctcagacagagagatggaaatcaagaggggaagagttacctcagcctctggatccctcacagcccatgctccctcccttccactcagagagtggcgctttggggatgctaaggatgcgcctccgtgtacttctaaggtgggagggggatacaagcagaggagaatatcggacgctcagacgttccattctgcctgccgctcttctctggttccactagggcttgtccttgtaagaaactgacggagcctagggcagctgtgagaggaagaggctggggcgcctggaacccgaacactcttgagtgctctcagttacagcctaccgagtccgcagcaagcattcagaaccatggacagcagcgccggcccagggaacatcagcgactgctctgaccccttagctcctgcaagttgctccccagcacctggctcctggctcaacttgtcccacgttgatggcaaccagtccgacccatgcggtcctaaccgcacggggcttggcgggagccacagcctgtgccctcagaccggcagcccttccatggtcacagccatcaccatcatggccctctattctatcgtgtgtgtagtgggcctctttggaaacttcctggtcatgtatgtgattgtaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcacgttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagctagaaaatctggaagcagaaactgctccattgccctaactgggtcccacgcc

SEQ ID NO: 17 - DNA, 1569 bp, Mus Sp.
ggcgcgggatctgggccgatgatggaagctttctctaagtctgcattccaaaagctcagacagagagatggaaatcaagaggggaagagttacctcaggtcttgtgcaggtgcactgctgctgtgaattcatgaagacaacaccctcccctttagaagacagtgcttcacaacactcccaactagcctctggctctgatgttcactttgtcccctcttctgaagcagggcttgtccttgtaagaaactgacggagcctagggcagctgtgagaggaagaggctggggcgcctggaacccgaacactcttgagtgctctcagttacagcctaccgagtccgcagcaagcattcagaaccatggacagcagcgccggcccagggaacatcagcgactgctctgaccccttagctcctgcaagttgctccccagcacctggctcctggctcaacttgtcccacgttgatggcaaccagtccgacccatgcggtcctaaccgcacggggcttggcgggagccacagcctgtgccctcagaccggcagcccttccatggtcacagccatcaccatcatggccctctattctatcgtgtgtgtagtgggcctctttggaaacttcctggtcatgtatgtgattgtaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcacgttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagctagaaaatctggaagcagaaactgctccattgccctaactgggtcccacgcc

SEQ ID NO: 18 - DNA, 1133 bp, Mus Sp.
ggcgcgggatctgggccgatgatggaagctttctctaagtctgcattccaaaagctcagacagagagatggaaatcaagaggggaagagttacctcaggctccctctgtccattcttttcctgaacaaagagtcatgacaactcaaagaatcaactgaaaatcaaaatagaaaatgggctaaggcaactggtcgaccaccacaaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcacgttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagctagaaaatctggaagcagaaactgctccattgccctaactgggtcccacgcc

SEQ ID NO: 19 - DNA, 1174 bp, Mus Sp.
ggcgcgggatctgggccgatgatggaagctttctctaagtctgcattccaaaagctcagacagagagatggaaatcaagaggggaagagttacctcagacacctcattccaaggaaggaaattatctttttaaaactgaaataactaggcattccaagcacctggcggtgagctgataaagactgagagtgtaatgagtcagaaaattgtgttgggttcccctcttgagtgtgactaatgtcaaaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcacgttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagctagaaaatctggaagcagaaactgctccattgccctaactgggtcccacgcc

SEQ ID NO: 20 - DNA, 1082 bp, Mus Sp.
ggcgcgggatctgggccgatgatggaagctttctctaagtctgcattccaaaagctcagacagagagatggaaatcaagaggggaagagttacctcagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcacgttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagaggaatgaggaaccttcttcctgatgatggcccaagacaggaatccggggaaggccagcttggcaggtgaatgtcatccgaacacagggatgagctggtgagcagtgtgg

SEQ ID NO: 21 - DNA, 2951 bp, Mus Sp.
ggaacccgaacactcttgagtgctctcagttacagcctaccgagtccgcagcaagcattcagaaccatggacagcagcgccggcccagggaacatcagcgactgctctgaccccttagctcctgcaagttgctccccagcacctggctcctggctcaacttgtcccacgttgatggcaaccagtccgacccatgcggtcctaaccgcacggggcttggcgggagccacagcctgtgccctcagaccggcagcccttccatggtcacagccatcaccatcatggccctctattctatcgtgtgtgtagtgggcctctttggaaacttcctggtcatgtatgtgattgtaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcacgttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagtgtgtatgagtgctatgcccacagggaccagaagatggtatcagaccttctagaactgaagtagtgagcagtccccacccccacccccccgcaatgtgagtagcttataaaatgattttatgtacttgttagctctccatggagcacaagataaaagtgacatcacagtttgaaataatagctctttgatcctagaatgaaagcatggaaaaaataagttgggtcatttgtctataggaaggaaggggacaaggtggggacagagaggactgagaagacgtagacaattaaggtaggaagaaggctaatctagatagcacatttacgttccaaatccactacttcttcttgtgtgtctttcaggcacaccaaaaacctcaagaatgcctgaaatgcagatgtctatcccttaccatcctggttatatgcctacatttccaacatcagcaattcttcataatgatcaaaaaaaatgtttcataactaaaggaaaaaccatctgcttcttttgatttaatgaaacttaaatatctctgggtgtgggggacattaggatgttaaagtttcttcaaaggaaagagataacttctcatagtgctgaaatgggtaccctcaagataggggacaggcaaacagagtttatggaagatgatattaagaaagaaaaacatatcaatcaagaaaaatagtgttacgtattttgacaacaaagcctaattgataacttacagaaattaatatatgtagaatgggataagacttctgtgcattgatgataaatctgctgcttagcccctgttacaatgtacagctaagtacgtctttcttgtctttctttctgtgctttcttcactttgatttaggctaaaatgtcagttattcaaaggcccctaatattgccaaatccagtctcattccagatcctgtagaattaatattagtttgagttgctctttcagagaaaatgacatgcagcccgaatcattattcacaaagaaaaagggccaatccaaggtgaagtgttgctaacactggaaaggtctgaacaaggcctactttcctaacaataacaacgcctcaagagatcttcaggatgaaatacaactcgaaaaatataaattataaagccctggacgtaaatcacaaggagtaagaggagtctctgacatattgggtaagatagagccccaagattaatgggaaagattctagcaaacgaacaaccacaaactatcaagctgtgtaaacttgtcccagaacctgggtcacagtgagaggagcaggtggctctgagaagcaagactgcatctggcaaaattgcaaagaaagaaattagctactagatggcacaattggatgaactcgagaacccagtggtttatgtagatttgaaaacctctatcaatctctgtaaccatacactgtgtttagttctgatctaaatttaatgatgctatgacttagctttataaaattttatctcattgtattcttaggagcctcagtcagcagagacatgatgtgaatgaacggactgattagacaaggtttcctgaacactgaaatacaaaacaaatagagagcttactagagaaaattcgtagcccgaaaattcaattatagaaacaaatgagtgttagagtagatatggtaaggcctcagagaggttttatttcatgactaacaacatgacccaaggcacctaatccatggtgattagattacaa

SEQ ID NO: 22 - DNA, 1332 bp, Mus Sp.
ggaacccgaacactcttgagtgctctcagttacagcctaccgagtccgcagcaagcattcagaaccatggacagcagcgccggcccagggaacatcagcgactgctctgaccccttagctcctgcaagttgctccccagcacctggctcctggctcaacttgtcccacgttgatggcaaccagtccgacccatgcggtcctaaccgcacggggcttggcgggagccacagcctgtgccctcagaccggcagcccttccatggtcacagccatcaccatcatggccctctattctatcgtgtgtgtagtgggcctctttggaaacttcctggtcatgtatgtgattgtaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcacgttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagacaagcctcacacttcagtaatggaatgagtagattaaatcggcgagcaagatggtgggaggagtcaaaatattttcatgccttcctgtggaactccaaaggaagacc

SEQ ID NO: 23 - DNA, 2588 bp, Mus Sp.
ggaacccgaacactcttgagtgctctcagttacagcctaccgagtccgcagcaagcattcagaaccatggacagcagcgccggcccagggaacatcagcgactgctctgaccccttagctcctgcaagttgctccccagcacctggctcctggctcaacttgtcccacgttgatggcaaccagtccgacccatgcggtcctaaccgcacggggcttggcgggagccacagcctgtgccctcagaccggcagcccttccatggtcacagccatcaccatcatggccctctattctatcgtgtgtgtagtgggcctctttggaaacttcctggtcatgtatgtgattgtaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcacgttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccaggcacaccaaaaacctcaagaatgcctgaaatgcagatgtctatcccttaccatcctggttatatgcctacatttccaacatcagcaattcttcataatgatcaaaaaaaatgtttcataactaaaggaaaaaccatctgcttcttttgatttaatgaaacttaaatatctctgggtgtgggggacattaggatgttaaagtttcttcaaaggaaagagataacttctcatagtgctgaaatgggtaccctcaagataggggacaggcaaacagagtttatggaagatgatattaagaaagaaaaacatatcaatcaagaaaaatagtgttacgtattttgacaacaaagcctaattgataacttacagaaattaatatatgtagaatgggataagacttctgtgcattgatgataaatctgctgcttagcccctgttacaatgtacagctaagtacgtctttcttgtctttctttctgtgctttcttcactttgatttaggctaaaatgtcagttattcaaaggcccctaatattgccaaatccagtctcattccagatcctgtagaattaatattagtttgagttgctctttcagagaaaatgacatgcagcccgaatcattattcacaaagaaaaagggccaatccaaggtgaagtgttgctaacactggaaaggtctgaacaaggcctactttcctaacaataacaacgcctcaagagatcttcaggatgaaatacaactcgaaaaatataaattataaagccctggacgtaaatcacaaggagtaagaggagtctctgacatattgggtaagatagagccccaagattaatgggaaagattctagcaaacgaacaaccacaaactatcaagctgtgtaaacttgtcccagaacctgggtcacagtgagaggagcaggtggctctgagaagcaagactgcatctggcaaaattgcaaagaaagaaattagctactagatggcacaattggatgaactcgagaacccagtggtttatgtagatttgaaaacctctatcaatctctgtaaccatacactgtgtttagttctgatctaaatttaatgatgctatgacttagctttataaaattttatctcattgtattcttaggagcctcagtcagcagagacatgatgtgaatgaacggactgattagacaaggtttcctgaacactgaaatacaaaacaaatagagagcttactagagaaaattcgtagcccgaaaattcaattatagaaacaaatgagtgttagagtagatatggtaaggcctcagagaggttttatttcatgactaacaacatgacccaaggcacctaatccatggtgattagattacaa

SEQ ID NO: 24 - DNA, 1695 bp, Mus Sp.
ggaacccgaacactcttgagtgctctcagttacagcctaccgagtccgcagcaagcattcagaaccatggacagcagcgccggcccagggaacatcagcgactgctctgaccccttagctcctgcaagttgctccccagcacctggctcctggctcaacttgtcccacgttgatggcaaccagtccgacccatgcggtcctaaccgcacggggcttggcgggagccacagcctgtgccctcagaccggcagcccttccatggtcacagccatcaccatcatggccctctattctatcgtgtgtgtagtgggcctctttggaaacttcctggtcatgtatgtgattgtaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcacgttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagataatgaaatttgaagctatctaccctaaactgagcttcaaatcttgggcattgaaatattttactttcattagagaaaagaaaaggaacacaaaagctggggctcttcctcccctccctacgtgtcatgctggatctccctcccaggctcacaggggcgtggctgcttggttgcttcctctaagacacatggggccttcttatccttcctgacccacctgaccttcctctaatggaagcagagccccaagctctctattccagcacatccctgttttaaacatagctgcccttcagattctctaacgctgccttcgactcccttccacacccagtgtttgcatgtcagtgtggatcttgcacagccagagagtagaagggaagataattggagaagctctgtcctaagtaactaaaaggtttgctttaaaaatacatccaattagcacttatcattatcactgcctctgc

SEQ ID NO: 25 - DNA, 1464 bp, Homo Sapiens
cggtgctcctggctacctcgcacagcgtgcccgcccggccgtcagtaccatggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcagtgcctacctataccttccctgtcttgctgggctctagagcatggctgcttggttgtgtaccctggaccactgcaaggacctcttgtcagatatgacctcccagctatccttcactcgtcctgccttcgtggaaatactgctcccagcccgtctggtggggcatttctcttgagttaagcatgattattcttcagttgcccgacactgcccttgactcccttccaaattcggcattttcacatcagtatggc

SEQ ID NO: 26 - DNA, 1388 bp, Homo Sapiens
cggtgctcctggctacctcgcacagcgtgcccgcccggccgtcagtaccatggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcagccacccttggcagtcagcatggcccagatctttacacgatatcctcctccgactcatcgtgagaaaacctgcaatgattacatgaagaggtagataatgtattaccctgatttggtaggtaaagtattatcctgatttatgtgacagagtgaaaggcaacttttaattgttaacc

SEQ ID NO: 27 - DNA, 1258 bp, Mus Sp.
ttttactgtccttgagaatggagaggatcagcaaagctgggaagccctccaggctcatttcagagagaatattccacagagcttgaaggcgcgggatctgggccgatgatggaagctttctctaagtctgcattccaaaagctcagacagagagatggaaatcaagaggggaagagttacctcagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcacgttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagccaaccctggcagtcagcgtggcccagatctttacaggatatccttctccgactcatgttgaaaaaccctgcaagagttgcatggacagaggaatgaggaaccttcttcctgatgatggcccaagacaggaatccggggaaggccagcttggcaggtgaatgtcatccgaacacagggatgagctggtgagcagtgtgg

SEQ ID NO: 28 - protein, 84 aa, Mus Sp.
MMEAFSKSAFQKLRQRDGNQEGKSYLRACPCKKLTEPRAAVRGRGWGAWNPNTLECSQLQPTESAASIQNHGQQRRPREHQRLL

SEQ ID NO: 29 - protein, 48 aa, Mus Sp.
MMEAFSKSAFQKLRQRDGNQEGKSYLSLWIPHSPCSLPSTQRVALWGC

SEQ ID NO: 30 - protein, 35 aa, Mus Sp.
MMEAFSKSAFQKLRQRDGNQEGKSYLRSCAGALLL

SEQ ID NO: 31 - protein, 39 aa, Mus Sp.
MMEAFSKSAFQKLRQRDGNQEGKSYLRLPLSILFLNKES

SEQ ID NO: 32 - protein, 41 aa, Mus Sp.
MMEAFSKSAFQKLRQRDGNQEGKSYLRHLIPRKEIIFLKLK

SEQ ID NO: 33 - protein, 370 aa, Mus Sp.
MMEAFSKSAFQKLRQRDGNQEGKSYLRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQPTLAVSVAQIFTGYPSPTHVEKPCKSCMDRGMRNLLPDDGPRQESGEGQLGR

SEQ ID NO: 34 - protein, 325 aa, Mus Sp.
MMEAFSKSAFQKLRQRDGNQEGKSYLRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQRNEEPSS

SEQ ID NO: 35 - protein, 388 aa, Mus Sp.
MDSSAGPGNISDCSDPLAPASCSPAPGSWLNLSHVDGNQSDPCGPNRTGLGGSHSLCPQTGSPSMVTAITIMALYSIVCVVGLFGNFLVMYVIVRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQCV

SEQ ID NO: 36 - protein, 392 aa, Mus Sp.
MDSSAGPGNISDCSDPLAPASCSPAPGSWLNLSHVDGNQSDPCGPNRTGLGGSHSLCPQTGSPSMVTAITIMALYSIVCVVGLFGNFLVMYVIVRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQTSLTLQ

SEQ ID NO: 37 - protein, 425 aa, Mus Sp.
MDSSAGPGNISDCSDPLAPASCSPAPGSWLNLSHVDGNQSDPCGPNRTGLGGSHSLCPQTGSPSMVTAITIMALYSIVCVVGLFGNFLVMYVIVRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQAHQKPQECLKCRCLSLTILVICLHFQHQQFFIMIKKNVS

SEQ ID NO: 38 - protein, 456 aa, Mus Sp.
MDSSAGPGNISDCSDPLAPASCSPAPGSWLNLSHVDGNQSDPCGPNRTGLGGSHSLCPQTGSPSMVTAITIMALYSIVCVVGLFGNFLVMYVIVRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQIMKFEAIYPKLSFKSWALKYFTFIREKKRNTKAGALPPLPTCHAGSPSQAHRGVAAWLLPLRHMGPSYPS

SEQ ID NO: 39 - protein, 446 aa, Homo Sapiens [sequence omitted]

SEQ ID NO: 40 - protein, 418 aa, Homo Sapiens
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQPPLAVSMAQIFTRYPPPTHREKTCNDYMKR

SEQ ID NO: 41 - protein, 22 aa, Mus Sp.
MEAFSKSAFQKLRQRDGNQEGC

SEQ ID NO: 42 - DNA, 210 bp, Mus Sp.
tttactgtccttgagaatggagaggatcagcaaagctgggaagccctccaggctcatttcagagagaatattccacagagcttgaaggcgcgggatctgggccgatgatggaagctttctctaagtctgcattccaaaagctcagacagagagatggaaatcaagaggggaagagttacctcaggttggtttctcttcagactgtagtga

SEQ ID NO: 43 - DNA, 179 bp, Mus Sp.
aaaatgaaatattgagaggtcagtctcttgcaggtcttgtgcaggtgcactgctgctgtgaattcatgaagacaacaccctcccctttagaagacagtgcttcacaacactcccaactagcctctggctctgatgttcactttgtcccctcttctgaagcaggtatttattacagtgtc

SEQ ID NO: 44 - DNA, 334 bp, Mus Sp.
tccttctctctcctccctccctctagcctctggatccctcacagcccatgctccctcccttccactcagagagtggcgctttggggatgctaaggatgcgcctccgtgtacttctaaggtgggagggggatacaagcagaggagaatatcggacgctcagacgttccattctgcctgccgctcttctctggttccactagggcttgtccttgtaagaaactgacggagcctagggcagctgtgagaggaagaggctggggcgcctggaacccgaacactcttgagtgctctcagttacagcctaccgagtccgcagcaagcattcagaaccatg

SEQ ID NO: 45 - DNA, 209 bp, Mus Sp.
agtaaacactaatcaaattttatttcacagacacctcattccaaggaaggaaattatctttttaaaactgaaataactaggcattccaagcaatggcggtgagctgataaagactgagagtgtaatgagtcagaaaattgtgttgggttcccctcttgagtgtgactaatgtcaaaaggtatgcctttgaatcactggtctatctttgc

SEQ ID NO: 46 - DNA, 170 bp, Mus Sp.
gattgggggatatattcttcatgctttcaggctccctctgtccattcttttcctgaacaaagagtcatgacaactcaaagaatcaactgaaaatcaaaatagaaaatgggctaaggcaactggtcgaccaccacaaaggtagttcattctctcaagcctcttttaccctt

SEQ ID NO: 47 - DNA, 19 bp, Mus Sp.
cgccccagcctcttcctct

SEQ ID NO: 48 - DNA, 24 bp, Mus Sp.
gacagtcactggtgcctatgcaat

SEQ ID NO: 49 - DNA, 23 bp, Mus Sp.
ctcttcccctcttgatttccatc

SEQ ID NO: 50 - DNA, 24 bp, Mus Sp.
gacgatcacgtgtgctcatgacat

SEQ ID NO: 51 - DNA, 1373 bp, Mus Sp.
ggaacccgaacactcttgagtgctctcagttacagcctaccgagtccgcagcaagcattcagaaccatggacagcagcgccggcccagggaacatcagcgactgctctgaccccttagctcctgcaagttgctccccagcacctggctcctggctcaacttgtcccacgttgatggcaaccagtccgacccatgcggtcctaaccgcacggggcttggcgggagccacagcctgtgccctcagaccggcagcccttccatggtcacagccatcaccatcatggccctctattctatcgtgtgtgtagtgggcctctttggaaacttcctggtcatgtatgtgattgtaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcacgttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagccaaccctggcagtcagcgtggcccagatctttacaggatatccttctccgactcatgttgaaaaaccctgcaagagttgcatggacaggtgagtgtgacccggactcaggtgacaaaataaaaggcaagttttagctttttgcacggc

SEQ ID NO: 52 - protein, 416 aa, Mus Sp.
MDSSAGPGNISDCSDPLAPASCSPAPGSWLNLSHVDGNQSDPCGPNRTGLGGSHSLCPQTGSPSMVTAITIMALYSIVCVVGLFGNFLVMYVIVRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQPTLAVSVAQIFTGYPSPTHVEKPCKSCMDR

SEQ ID NO: 53 - protein, 50 aa, Mus Sp.
MMEAFSKSAFQKLRQRDGNQEGKSYLRYTKMKTATNHQLENLEAETAPLP

(19) United States
(12) Patent Application Publication
(10) Pub. No.: US 2003/0068805 A1
(43) Pub. Date: Apr. 10, 2003

(54) Endo beta-1,4-glucanase from aspergillus

(76) Inventors: Susan Madrid, Vedbaek (DK); Preben Rasmussen, Kirke Hyllinge (DK); Anita Baruch, Glostrup (DK)

Correspondence Address: FROMMER LAWRENCE & HAUG, 745 FIFTH AVENUE- 10TH FL., NEW YORK, NY, 10151, US

(21) Appl. No.: 10/194,595

(22) Filed: Jul. 11, 2002

Related U.S. Application Data
(62) Division of application No. 09/561,523, filed on Apr. 28, 2000.
Application No. 09/561,523 is a continuation-in-part of application No. 08/913,264, filed on Apr. 6, 1998, now Pat. No. 6,077,702.
Application No. 08/913,264 is a 371 of international application No. PCT/EP96/01008, filed on Mar. 11, 1996.

(30) Foreign Application Priority Data
Mar. 17, 1995 (GB) 9505475.5

(51) Int. Cl. C12N 9/24; C12N 1/16; C07H 21/04; C12N 15/74; C12P 21/02

(52) U.S. Cl. 435/200; 435/69.1; 435/320.1; 435/254.3; 536/23.2

(57) ABSTRACT

A glucanase enzyme is described. In addition, there is described a nucleotide sequence coding for the glucanase enzyme and a promoter for controlling its expression.

The present invention relates to an enzyme. In addition, the present invention relates to a nucleotide sequence coding for the enzyme. Also, the present invention relates to a promoter, wherein the promoter can be used to control the expression of the nucleotide sequence coding for the enzyme.

In particular, the enzyme of the present invention is a glucanase enzyme—i.e. an enzyme that can degrade β-1,4-glucosidic bonds.

It is known that it is desirable to direct expression of a gene of interest (“GOI”) in certain tissues of an organism—such as a filamentous fungus (such as Aspergillus niger) or even a plant crop. The resultant protein or enzyme may be useful for the organism itself. For example, it may be desirable to produce crop protein products with an optimised amino acid composition and so increase the nutritive value of a crop. For example, the crop may be made more useful as a feed. In the alternative, it may be desirable to isolate the resultant protein or enzyme and then use the protein or enzyme to prepare, for example, food compositions. In this regard, the resultant protein or enzyme can be a component of the food composition or it can be used to prepare food compositions, including altering the characteristics or appearance of food compositions.

It may even be desirable to use the organism, such as a filamentous fungus or a crop plant, to express non-plant genes, such as for the same purposes.

Also, it may be desirable to use an organism, such as a filamentous fungus or a crop plant, to express mammalian genes. Examples of the latter products include interferons, insulin, blood factors and plasminogen activators

It is also desirable to use micro-organisms, such as filamentous fungi, to prepare products from GOIs by use of promoters that are active in the micro-organisms.

Fruit and vegetable cell walls largely consist of polysaccharide, the major components being pectin, cellulose and xyloglucan, R. R. Selvendran and J. A. Robertson, IFR Report 1989. Numerous cell wall models have been proposed which attempt to incorporate the essential properties of strength and flexibility (P. Albersheim, Sci. Am. 232, 81-95, 1975;, P. Albersheim, Plant Biochem. 3rd Edition (Bonner and Varner), Ac. Press, 1976; T. Hayashi, Ann. Rev. Plant Physiol. & Plant Mol. Biol., 40, 139-168, 1989).

The composition of the plant cell wall is complex and variable. Polysaccharides are mainly found in the form of long chains of cellulose (the main structural component of the plant cell wall), hemicellulose (comprising various β-xylan chains, such as xyloglucans) and pectic substances (consisting of galacturonans and rhamnogalacturonans, arabinans; and galactans and arabinogalactans).

In particular, glucans are polysaccharides made up exclusively of glucose subunits. Typical examples of glucans are starch and cellulose.

The enzymes that degrade glucans are collectively referred to as glucanases. A typical glucanase is β-1,4-endoglucanase.

β-1,4-endoglucanases have uses in many industries. For example, in the brewing industry, barley is used for production of malt, and, in the latter years, as adjunct in the brewing process. When the quality of the malt is poor, or barley has been used as an adjunct, problems with high viscosity in the wort can arise because of β-glucans from the barley. In this regard, barley contains large quantities of mixed β-1,3/1,4- glucans of very high molecular weight. When dissolved, these glucans produce high viscosity solutions, which can cause troubles in some applications. For example, the high viscosity reduces the filterability of the won and can lead to unacceptable long filtration times. To avoid these problems β-glucanase has been traditionally added to wort to avoid such problems—i.e. the problem with glucans can be avoided by addition of enzymes, in particular, glucanases, which degrade the polymers.

Further information on these problems may be found in the Grindsted brochure called “Glucanase GV”, the reviews by Dr. C. W. Bamforth (Brewers Digest June 1982 pages 22-28; and Brewers' Guardian September 1985 pages 21-26), and the paper by T. Godfrey (Industrial Enzymology The Application of Enzymes in Industry Chapter 4.5 pages 221-259).

In the feed industry barley can be used for chicken feed because it is cheap, but again the β-glucan can give problems for the digestion of the chicken. By addition of β-glucanase to the feed the digestibility of the feed can be increased. In addition, the faeces of chickens feeding on feed containing barley is sticky making it difficult to remove and results in dirty eggs.

WO 93/2019 discusses endo-β-1,4-glucanases (EC no. 3.2.1.4). According to WO 93/2019, these glucanases are a group of hydrolases which catalyse endo hydrolysis of 1,4-β-D-glycosidic linkages in cellulose, lichenin, cereal β-D-glucans and other plant material containing cellulosic parts. Endo-1,4-β-D-glucan 4-glucano hydrolase is sometimes called endo-β-1,4-glucanase.

The endo-β-1,4-glucanase of WO 93/2019 exhibits a pH-optimum of 2.0 to 4.0, an isoelectric point of 2.0 to 3.5, a molecular weight of between 30,000 and 50,000, and a temperature optimum between 30 and 70° C.

Further teachings on glucans may be found in WO 93/17101, in particular xyloglucans. According to WO 93/17101 the xyloglucans are 1,4-β-glucans that have been extensively substituted with α-1,6-xylosyl side chains, some of which are 1,2-β-galactosylated. They are found in large amounts in the primary cell walls of dicots but also in certain seeds, where they serve different roles. Primary cell wall xyloglucan is fucosylated. Xyloglucan is tightly hydrogen bonded to cellulose microfibrils and requires concentrated alkali or strong swelling agents to release it. Xyloglucan is thought to form cross-bridges between cellulose microfibrils, the cellulose/xyloglucan network forming the major load-bearing/elastic network of the wall. DCB mutated suspension culture cells (cell walls lacking cellulose) release xyloglucan into their media, suggesting that xyloglucan is normally rightly bound to cellulose.

Hydrolysis of primary cell wall xyloglucan has been demonstrated in segments of dark grown squash hypocotyls, during IAA induced growth (K. Wakabayashi et al, Plant Physiol., 95, 1070-1076, 1991). Endohydrolysis of wall xyloglucan is thought to contribute to wall loosening which accompanies cell expansion (T. Hyashi, Ann. Rev. Plant Physiol. & Plant Mol. Biol., 40, 139-168, 1989). The average molecular weight of xyloglucan has also been shown to decrease during tomato fruit ripening and this may contribute to the tissue softening which accompanies the ripening process (D. J. Huber, J. Amer. Soc. Hort. Sci., 108(3), 405-409, 1983). Certain seeds, e.g. Nasturtium, contain up to 30% by weight of xyloglucan, stored in thickened cotyledonary cell walls, which serves as a reserve polysaccharide and is rapidly depolymerised during germination.

It would be useful to increase glucanase activity, for example to have a plant with high concentration of glucanase for use in feed, preferably using recombinant DNA techniques.

The present invention seeks to provide an enzyme having glucanase activity; preferably wherein the enzyme can be prepared in certain or specific cells or tissues, such as in just a specific cell or tissue, of an organism, typically a filamentous fungus, preferably of the genus Aspergillus, such as Aspergillus niger, or even a plant.

Also, the present invention seeks to provide a GOI coding for the enzyme that can be expressed preferably in specific cells or tissues, such as in certain or specific cells or tissues, of an organism, typically a filamentous fungus, preferably of the genus Aspergillus, such as Aspergillus niger, or even a plant.

In addition, the present invention seeks to provide a promoter that is capable of directing expression of a GOI, such as a nucleotide sequence coding for the enzyme according to the present inventions preferably in certain specific cells or tissues, such as in just a specific cell or tissue, of an organism, typically a filamentous fungus, preferably of the genus Aspergillus, such as Aspergillus niger, or even a plant. Preferably, the promoter is used in Aspergillus wherein the product encoded by the GOI is excreted from the host organism into the surrounding medium.

Furthermore, the present invention seeks to provide constructs, vectors, plasmids, cells, tissues, organs and organisms comprising the GOI and/or the promoter, and methods of expressing the same, preferably in specific cells or tissues, such as expression in just a specific cell or tissue, of an organism, typically a filamentous fungus, preferably of the genus Aspergillus, or even a plant.

According to a first aspect of the present invention there is provided an enzyme obtainable from Aspergillus, wherein the enzyme has the following characteristics: a-MW of 24,235 D±50 D; a pI value of about 4; glucanase activity; and wherein the glucanase activity is endo β-1,4-glucanase activity.

According to a second aspect of the present invention there is provided an enzyme having the sequence shown as SEQ. I.D. No. 1 or a variant, homologue or fragment thereof.

According to a third aspect of the present invention there is provided an enzyme coded by the nucleotide sequence shown as SEQ. I.D. No. 2 or a variant, homologue or fragment thereof or a sequence complementary thereto.

According to a fourth aspect of the present invention there is provided a nucleotide sequence coding for the enzyme according to the present invention.

According to a fifth aspect of the present invention there is provided a nucleotide sequence having the sequence shown as SEQ. I.D. No. 2 or a variant, homologue or fragment thereof or a sequence complementary thereto.

According to a sixth aspect of the present invention there is provided a promoter having the sequence shown as SEQ. I.D. No. 3 or a variant, homologue or fragment thereof or a sequence complementary thereto.

According to a seventh aspect of the present invention there is provided a terminator having the nucleotide sequence shown as SEQ. I.D. No. 13 or a variant, homologue or fragment thereof or a sequence complementary thereto.

According to an eighth aspect of the present invention there is provided a signal sequence having the nucleotide sequence shown as SEQ. I.D. No. 14 or a variant, homologue or fragment thereof or a sequence complementary thereto.

According to a ninth aspect of the present invention there is provided a process for expressing a GOI by use of a promoter, wherein the promoter is the promoter according to the present invention.

According to a tenth aspect of the present invention there is provided the use of an enzyme according to the present invention to degrade a glucan.

According to an eleventh aspect of the present invention there is provided plasmid NCIMB 40704, or a nucleotide sequence obtainable therefrom for expressing an enzyme capable of degrading arabinoxylan or for controlling the expression thereof or for controlling the expression of another GOI.

According to a twelfth aspect of the present invention there is provided a signal sequence having the sequence shown as SEQ. I.D. No. 15 or a variant, homologue or fragment thereof.

According to a thirteenth aspect of the present invention there is provided a glucanase enzyme having the ability to degrade β-1,4-glucosidic bonds, which is immunologically reactive with an antibody raised against a purified glucanase enzyme having the sequence shown as SEQ. I.D. No. 1.

According to a fourteenth aspect of the present invention there is provided a promoter that is inducible by glucose.

According to a fifteenth aspect of the present invention there is provided the use of glucose to induce a promoter.

Other aspects of the present invention include constructs, vectors, plasmids, cells, tissues, organs and transgenic organisms comprising the aforementioned aspects of the present invention.

Other aspects of the present invention include methods of expressing or allowing expression or transforming any one of the nucleotide sequence, the construct, the plasmid, the vector, the cell, the tissue, the organ or the organism, as well as the products thereof.

Additional aspects of the present invention include uses of the promoter for expressing GOIs in culture media such as a broth or in a transgenic organism.

Further aspects of the present invention include uses of the enzyme for preparing or treating foodstuffs, including animal feed.

In the following text, the enzyme of the present invention is sometimes referred to as Egla enzyme and the coding sequence therefor is sometimes referred to as the Egla gene. In addition, the promoter of the present invention is sometimes referred to as Egla promoter.

Preferably the enzyme is coded by the nucleotide sequence shown as SEQ. I.D. No. 2 or a variant, homologue or fragment thereof or a sequence complementary thereto.

Preferably the nucleotide sequence has the sequence shown as SEQ. I.D. No. 2 or a variant, homologue or fragment thereof or a sequence complementary thereto.

Preferably the nucleotide sequence is operatively linked to a promoter.

Preferably the promoter is the promoter having the sequence shown as SEQ. I.D. No. 3 or a variant, homologue or fragment thereof or a sequence complementary thereto.

Preferably the promoter of the present invention is operatively linked to a GOI.

Preferably the GOI comprises a nucleotide sequence according to the present invention.

In one preferred embodiment, the transgenic organism is a fungus. For example the organism can be a yeast, which would then be useful in for example the brewing industry.

Preferably the transgenic organism is a filamentous fungus, more preferably of the genus Aspergillus.

In another preferred embodiment the transgenic organism is a plant.

In another preferred embodiment the transgenic organism is a yeast. In this regard, yeast have been widely used as a vehicle for heterologous gene expression. The species Saccharomyces cerevisiae has a long history of industrial use, including use for heterologous gene expression. Expression of heterologous genes in Saccharomyces cerevisiae has been reviewed by Goodey et al (1987, Yeast Biotechnology, D R Berry et al, eds., pp 401-429, Allen and Unwin, London) and by King et al (1989, Molecular and Cell Biology of Yeasts, E F Walton and G T Yarronton, eds, pp 107-133, Blackie, Glasgow).

For several reasons Saccharomyces cerevisiae is well suited for heterologous gene expression. First, it is non-pathogenic to humans and incapable of producing certain endotoxins. Second, it has a long history of safe use following centuries of commercial exploitation for various purposes. This has led to wide public acceptability. Third, the extensive commercial use and research devoted to the organism has resulted in a wealth of knowledge about the genetics and physiology as well as large-scale fermentation characteristics of Saccharomyces cerevisiae.

An additional advantage is that yeasts are capable of post-translational modifications of proteins and thereby have the potential for glycosylation and/or secretion of heterologous gene products into the growth medium. A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol 5, Anthony H Rose and J Stuart Harrison, eds, 2nd edition, Academic Press Ltd.).

The glycosylation of enzymes expressed in yeast is known to increase heat stability of the enzyme. Enhancing the heat stability of the glucanase according to the present invention will make this enzyme suitable for use in the brewing industry and for use in the preparation of animal feed, i.e. chicken feed.

Yeasts are known to secrete very few proteins into the culture medium. This makes yeast a very attractive host for expression of heterologous genes, since secretable gene products can easily be recovered and purified.

Several types of yeast vectors are available, including integrative vectors, which require recombination with the host genome for their maintenance, and autonomously replicating plasmid vectors.

In order to prepare the transgenic Saccharomyces, expression constructs are prepared by inserting a GOI (such as an amylase or SEQ. ID No. 2) into a construct designed for expression in yeast. Several types of constructs used for heterologous expression have been developed. The constructs contain a promoter active in yeast fused to the GOI, usually a promoter of yeast origin, such as the GAL1 promoter, is used. The GOI can be fused to a signal sequence which directs the protein encoded by the GOI to be secreted. Usually a signal sequence of yeast origin, such as the sequence encoding the SUC2 signal peptide, is used. A terminator active in yeast ends the expression system.

Heterologous expression in yeast has been reported for several genes. The gene products can be glycosylated which is advantageous for some enzymes intended for specific application where heat tolerance is desirable. The proteins can be deposited intracellularly if the GOI is not fused to a signal sequence, or they can be secreted extracelluarly if the GOI is equipped with a signal sequence.

For the transformation of yeast several transformation protocols have been developed.

For example, the transgenic Saccharomyces according to the present invention can be prepared by following the teachings of Hinnen et al (1978, Proceedings of the National Academy of Sciences of the USA 75, 1929) Beggs, J D (1978, Nature, London, 275, 104); and Ito, H et al (1983, J Bacteriology 153, 163-168).

The transformed yeast cells are selected using various selective markers. Among the markers used for transformation are a number of auxotrophic markers such as LEU2, HIS4 and TRP1, and dominant antibiotic resistance markers such as aminoglycoside antibiotic markers, eg G418.

Highly preferred embodiments of each of the aspects of the present invention do not include any one of the native enzyme, the native promoter or the native nucleotide sequence in its natural environment.

Preferably, in any one of the plasmid, the vector such as an expression vector or a transformation vector, the cell, the tissue, the organ, the organism or the transgenic organism, the promoter is present in combination with at least one GOI.

Preferably the promoter and the GOI are stably incorporated within the transgenic organism's genome.

Preferably the transgenic organism is a filamentous fungus, preferably of the genus Aspergillus, more preferably Aspergillus niger. Alternatively, the transgenic organism can be a yeast. The transgenic organism can even be a plant, such as a monocot or dicot plant.

A highly preferred embodiment is an enzyme obtainable from Aspergillus, wherein the enzyme has the following characteristics: a MW of 24,235 D±50 D; a pI value of about 4; glucanase activity; and wherein the glucanase activity is endo β-1,4-glucanase activity; wherein the enzyme has the sequence shown as SEQ. I.D. No. 1 or a variant, homologue or fragment thereof.

Another highly preferred embodiment is an enzyme obtainable from Aspergillus, wherein the enzyme has the following characteristics: a MW of 24,235 D±50 D; a pI value of about 4; glucanase activity; and wherein the glucanase activity is endo β-1,4-glucanase activity; wherein the enzyme is coded by the nucleotide sequence shown as SEQ. I.D. No. 2 or a variant, homologue or fragment thereof or a sequence complementary thereto.

The advantages of the present invention are that it provides a means for preparing a glucanase enzyme and the nucleotide sequence coding for the same. In addition, it provides a promoter that can control the expression of that, or another, nucleotide sequence.

Other advantages of the present invention are that the enzyme can be used to prepare useful feeds containing cereals, such as barley, maize, rice etc.

The present invention therefore provides an enzyme having glucanase activity wherein the enzyme can be prepared in certain or specific cells or tissues, such as in just a specific cell or tissue, of an organism, typically a filamentous fungus, preferably of the genus Aspergillus, such as Aspergillus niger. The enzyme may even be prepared in a plant.

Also, the present invention provides a GOI coding for the enzyme that can be expressed preferably in specific cells or tissues, such as in certain or specific cells or tissues, of an organism, typically a filamentous fungus, preferably of the genus Aspergillus, such as Aspergillus niger. The GOI may even be expressed in a plant.

In addition, the present invention provides a promoter that is capable of directing expression of a GOI, such as a nucleotide sequence coding for the enzyme according to the present invention, preferably in certain specific cells or tissues, such as in just a specific cell or tissue, of an organism, typically a filamentous fungus, preferably of the genus Aspergillus, such as Aspergillus niger, or even a plant. Preferably, the promoter is used in Aspergillus wherein the product encoded by the GOI is excreted from the host organism into the surrounding medium. The promoter may even be tailored (if necessary) to express a GOI in a plant.

The present invention also provides constructs, vectors, plasmids, cells, tissues, organs and organisms comprising the GOI and/or the promoter, and methods of expressing the same, preferably in specific cells or tissues, such as expression in just a specific cell or tissue, of an organism, typically a filamentous fungus, preferably of the genus Aspergillus, or even a plant.

The terms “variant”, “homologue” or “fragment” in relation to the enzyme include any substitution of, variation of, modification of, replacement of, deletion of or addition of one (or more) amino acid from or to the sequence providing the resultant amino acid sequence has glucanase activity, preferably having at least the same activity of the enzyme shown in the sequence listings (SEQ I.D. No. 1 or 12). In particular, the term “homologue” covers homology with respect to structure and/or function providing the resultant enzyme has glucanase activity. With respect to sequence homology, preferably there is at least 75%, more preferably at least 85%, more preferably at least 90% homology to SEQ ID NO. 1 shown in the attached sequence listings. More preferably there is at least 95%, more preferably at least 98%, homology to SEQ ID NO. 1 shown in the attached sequence listings.

The terms “variant”, “homologue” or “fragment” in relation to the nucleotide sequence coding for the enzyme include any substitution of, variation of, modification of, replacement of, deletion of or addition of one (or more) nucleic acid from or to the sequence providing the resultant nucleotide sequence codes for an enzyme having glucanase activity, preferably having at least the same activity of the enzyme shown in the sequence listings (SEQ I.D. No. 2 or 12). In particular, the term “homologue” covers homology with respect to structure and/or function providing the resultant nucleotide sequence codes for an enzyme having glucanase activity. With respect to sequence homology, preferably there is at least 75 %, more preferably at least 85 %, more preferably at least 90% homology to SEQ ID NO. 2 shown in the attached sequence listings. More preferably there is at least 95%, more preferably at least 98%, homology to SEQ ID NO. 2 shown in the attached sequence listings.

The terms “variant” , “homologue” or “fragment” in relation to the promoter include any substitution of, variation of, modification of, replacement of, deletion of or addition of one (or more) nucleic acid from or to the sequence providing the resultant nucleotide sequence has the ability to act as a promoter in an expression system—such as the transformed cell or the transgenic organism according to the present invention. In particular, the term “homologue” covers homology with respect to structure and/or function providing the resultant nucleotide sequence has the ability to act as a promoter. With respect to sequence homology, preferably there is at least 75%, more preferably at least 85%, more preferably at least 90% homology to SEQ ID NO. 3 shown in the attached sequence listings. More preferably there is at least 95%, more preferably at least 98%, homology to SEQ ID NO. 3 shown in the attached sequence listings.

The terms “variant”, “homologue” or “fragment” in relation to the terminator or signal nucleotide sequences include any substitution of, variation of, modification of, replacement of, deletion of or addition of one (or more) nucleic acid from or to the sequence providing the resultant nucleotide sequence has the ability to act as a terminator or codes for an amino acid sequence that has the ability to act as a signal sequence respectively in an expression system—such as the transformed cell or the transgenic organism according to the present invention. In particular, the term “homologue” covers homology with respect to structure and/or function providing the resultant nucleotide sequence has the ability to act as or code for a terminator or signal respectively. With respect to sequence homology, preferably there is at least 75%, more preferably at least 85%, more preferably at least 90% homology to SEQ ID NO.s 13 and 14 (respectively) shown in the attached sequence listings. More preferably there is at least 95%, more preferably at least 98%, homology to SEQ ID NO.s 13 and 14 (respectively) shown in the attached sequence listings.

The terms “variant”, “homologue” or “fragment” in relation to the signal amino acid sequence include any substitution of, variation of, modification of, replacement of, deletion of or addition of one (or more) amino acid from or to the sequence providing the resultant sequence has the ability to act as a signal sequence in an expression system—such as the transformed cell or the transgenic organism according to the present invention. In particular, the term “homologue” covers homology with respect to structure and/or function providing the resultant nucleotide sequence has the ability to act as or code for a signal. With respect to sequence homology, preferably there is at least 75%, more preferably at least 85%, more preferably at least 90% homology to SEQ ID NO 15 shown in the attached sequence listings. More preferably there is at least 95%, more preferably at least 98%, homology to SEQ ID NO 15 shown in the attached sequence listings.

The above terms are synonymous with allelic variations of the sequences.

The term “complementary” means that the present invention also covers nucleotide sequences that can hybridise to the nucleotide sequences of the coding sequence, the promoter sequence, the terminator sequence or the signal sequence respectively.

The term “nucleotide” in relation to the present invention includes genomic DNA, CDNA, synthetic DNA, and RNA. Preferably it means DNA, more preferably cDNA for the coding sequence of the present invention since the genomic coding sequence has two introns and their removal would allow expression in bacteria.

The term “construct”—which is synonymous with terms such as “conjugate”, “cassette” and “hybrid”—includes a GOI directly or indirectly attached to a promoter. An example of an indirect attachment is the provision of a suitable spacer group such as an intron sequence, such as the Sh1-intron or the ADH intron, intermediate the promoter and the GOI. The same is true for the term “fused” in relation to the present invention which includes direct or indirect attachment. In each case, it is highly preferred that the terms do not cover the natural combination of the gene coding for the enzyme ordinarily associated with the wild type gene promoter and when they are both in their natural environment. A highly preferred embodiment is the or a GOI being operatively linked to a or the promoter.

The construct may even contain or express a marker which allows for the selection of the genetic construct in, for example, a filamentous fungus, preferably of the genus Aspergillus, such as Aspergillus niger, or plants, preferably cereals, such as maize, rice, barley etc., into which it has been transferred. Various markers exist which may be used, such as for example those encoding mannose-6-phosphate isomerase (especially for plants) or those markers that provide for antibiotic resistance—e.g. resistance to G418, hygromycin, bleomycin, kanamycin and gentamycin.

The term “vector” includes expression vectors and transformation vectors.

The term “expression vector” means a construct capable of in vivo or in vitro expression.

The term “transformation vector” means a construct capable of being transferred from one species to another—such as from an E. coli plasmid to a filamentous fungus, preferably of the genus Aspergillus. It may even be a construct capable of being transferred from an E. coli plasmid to an Agrobacterium to a plant.

The term “tissue” includes tissue per se and organ

The term “organism” in relation to the present invention includes any organism that could comprise the promoter according to the present invention and/or the nucleotide sequence coding for the enzyme according to the present invention and/or products obtained therefrom, wherein the promoter can allow expression of a GOI and/or wherein the nucleotide sequence according to the present invention can be expressed when present in the organism.

Preferably the organism is a filamentous fungus, preferably of the genus Aspergillus, more preferably Aspergillus niger.

The term “transgenic organism” in relation to the present invention includes any organism that comprises the promoter according to the present invention and/or the nucleotide sequence coding for the enzyme according to the present invention and/or products obtained therefrom, wherein the promoter can allow expression of a GOI and/or wherein the nucleotide sequence according to the present invention can be expressed within the organism. Preferably the promoter and/or the nucleotide sequence is (are) incorporated in the genome of the organism. Preferably the transgenic organism is a filamentous fungus, preferably of the genus Aspergillus, more preferably Aspergillus niger.

Therefore, the transgenic organism of the present invention includes an organism comprising any one of, or combinations of, the promoter according to the present invention, the nucleotide sequence coding for the enzyme according to the present invention, constructs according to the present invention, vectors according to the present invention, plasmids according to the present invention, cells according to the present invention, tissues according to the present invention or the products thereof. For example the transgenic organism can comprise a GOI, preferably an exogenous nucleotide sequence, under the control of the promoter according to the present invention. The transgenic organism can also comprise the nucleotide sequence coding for the enzyme of the present invention under the control of a promoter, which may be the promoter according to the present invention.

In a highly preferred embodiment, the transgenic organism does not comprise the combination of the promoter according to the present invention and the nucleotide sequence coding for the enzyme according to the present invention, wherein both the promoter and the nucleotide sequence are native to that organism and are in their natural environment. Thus, in these highly preferred embodiments, the present invention does not cover the native nucleotide coding sequence according to the present invention in its natural environment when it is under the control of its native promoter which is also in its natural environment. In addition, in this highly preferred embodiment, the present invention does not cover the native enzyme according to the present invention when it is in its natural environment and when it has been expressed by its native nucleotide coding sequence which is also in its natural environment and when that nucleotide sequence is under the control of its native promoter which is also in its natural environment.

The term “promoter” is used in the normal sense of the art, e.g. an RNA polymerase binding site in the Jacob-Mond theory of gene expression.

In one aspect, the promoter of the present invention is capable of expressing a GOI, which can be the nucleotide sequence coding for the enzyme of the present invention.

In another aspect, the nucleotide sequence according to the present invention is under the control of a promoter that allows expression of the nucleotide sequence. In this regard, the promoter need not necessarily be the same promoter as that of the present invention. In this aspect, the promoter may be a cell or tissue specific promoter. If, for example, the organism is a plant then the promoter can be one that affects expression of the nucleotide sequence in any one or more of stem, sprout, root and leaf tissues.

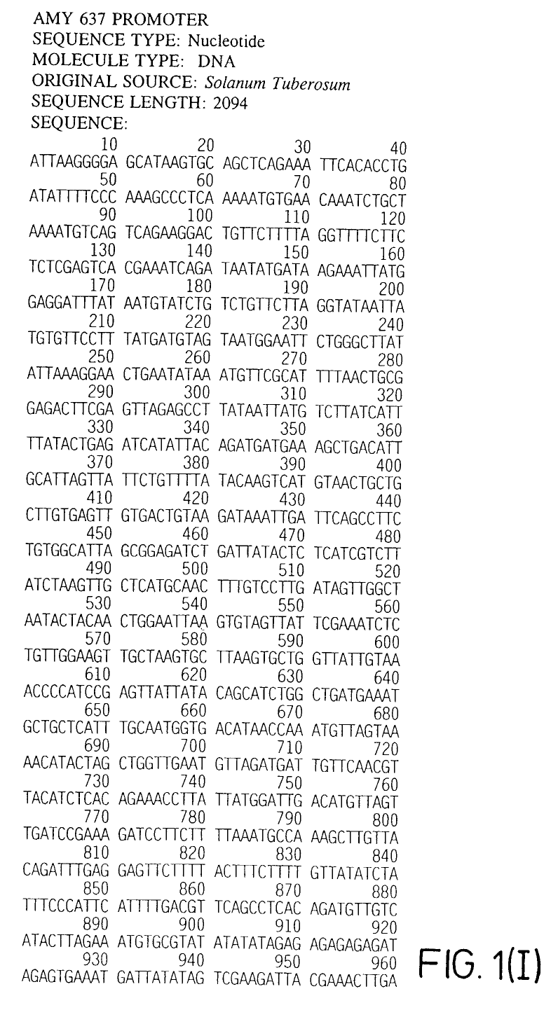

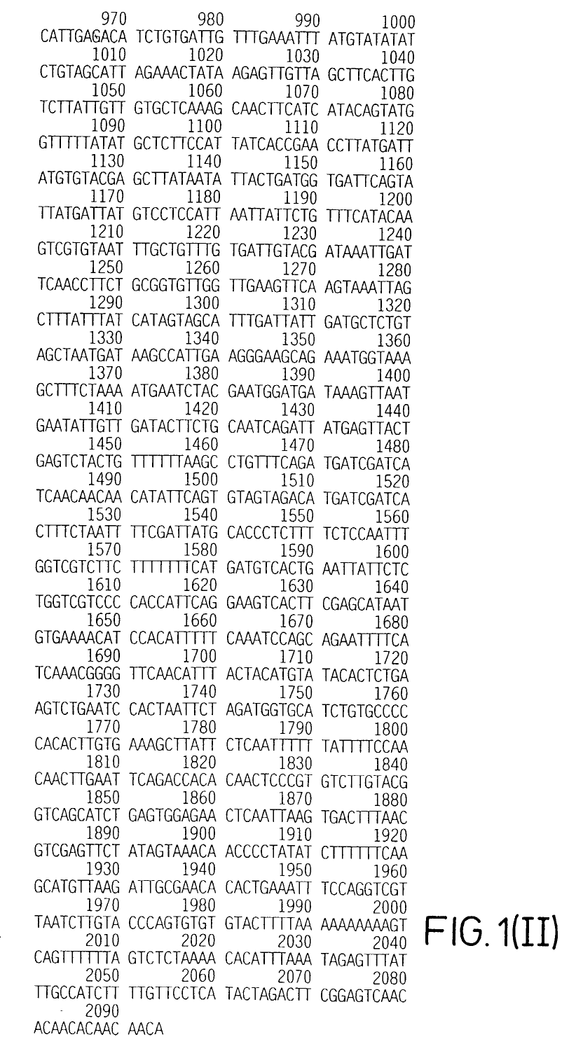

By way of example, the promoter for the nucleotide sequence of the present invention can be the α-Amy 1 promoter (otherwise known as the Amy 1 promoter, the Amy 637 promoter or the α-Amy 637 promoter) as described in our co-pending UK patent application No. 9421292.5 filed Oct. 21, 1994. That promoter comprises the sequence shown in FIG. 1.

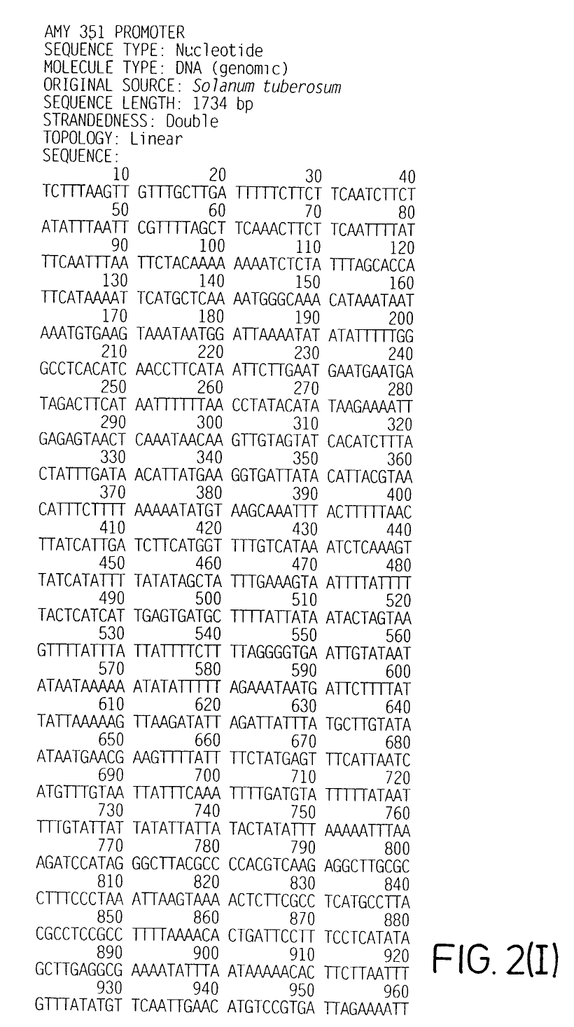

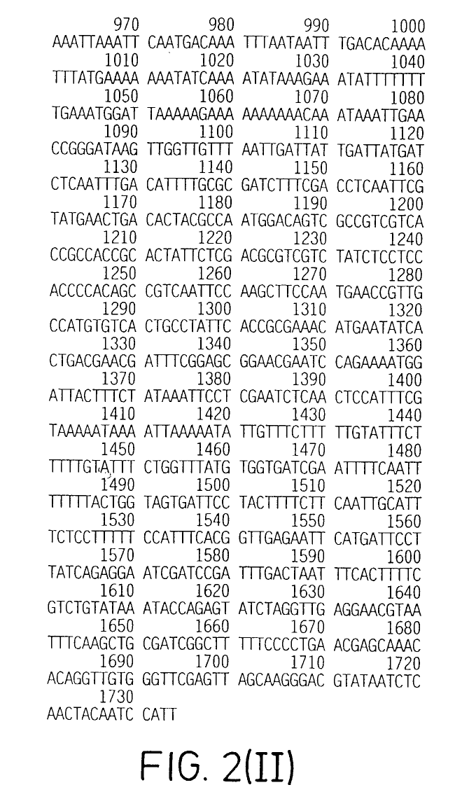

Alternatively, the promoter for the nucleotide sequence of the present invention can be the α-Amy 3 promoter (otherwise known as the Amy 3 promoter the Amy 351 promoter or the α-Amy 351 promoter) as described in our co-pending UK patent application No 9421286.7 filed Oct. 21, 1994. That promoter comprises the sequence shown in FIG. 2.

Preferably, the promoter is the promoter of the present invention

In addition to the nucleotide sequences described above, the promoters, particularly that of the present invention, could additionally include features to ensure or to increase expression in a suitable host. For example, the features can be conserved regions such as a Pribnow Box or a TATA box. The promoters may even contain other sequences to affect (such as to maintain, enhance, decrease) the levels of expression of the GOI. For example, suitable other sequences include the Sh1-intron or an ADH intron. Other sequences include inducible elements—such as temperature, chemical, light or stress inducible elements. Also, suitable elements to enhance transcription or translation may be present. An example of the latter element is the TMV 5′ signal sequence (see Sleat Gene 217 [1987] 217-225; and Dawson Plant Mol. Biol. 23 [1993] 97).

In addition the present invention also encompasses combinations of promoters and/or nucleotide sequences coding for proteins or enzymes and/or elements. For example, the present invention encompasses the combination of a promoter according to the present invention operatively linked to a GOI, which could be a nucleotide sequence according to the present invention, and another promoter such as a tissue specific promoter operatively linked to the same or a different GOI.

The present invention also encompasses the use of promoters to express a nucleotide sequence coding for the enzyme according to the present invention, wherein a part of the promoter is inactivated but wherein the promoter can still function as a promoter. Partial inactivation of a promoter in some instances is advantageous.

In particular, with the Amy 351 promoter mentioned earlier it is possible to inactivate a part of it so that the partially inactivated promoter expresses GOIs in a more specific manner such as in just one specific tissue type or organ.

The term “inactivated” means partial inactivation in the sense that the expression pattern of the promoter is modified but wherein the partially inactivated promoter still functions as a promoter. However, as mentioned above, the modified promoter is capable of expressing a GOI in at least one (but not all) specific tissue of the original promoter One such promoter is the Amy 351 promoter described above.

Examples of partial inactivation include altering the folding pattern of the promoter sequence, or binding species to parts of the nucleotide sequence, so that a part of the nucleotide sequence is not recognised by, for example, RNA polymerase. Another, and preferable, way of partially inactivating the promoter is to truncate it to form fragments thereof. Another way would be to mutate at least a part of the sequence so that the RNA polymerase can not bind to that part or another part.

Another modification is to mutate the binding sites for regulatory proteins for example the CreA protein known from filamentous fungi to exert carbon catabolite repression, and thus abolish the catabolite repression of the native promoter.

The term “GOI” with reference to the present invention means any gene of interest. A GOI can be any nucleotide that is either foreign or natural to the organism (e.g. filamentous fungus, preferably of the genus Aspergillus, or a plant) in question. Typical examples of a GOI include genes encoding for proteins and enzymes that modify metabolic and catabolic processes. The GOI may code for an agent for introducing or increasing pathogen resistance. The GOI may even be an antisense construct for modifying the expression of natural transcripts present in the relevant tissues. The GOI may even code for a non-natural protein of a filamentous fungus, preferably of the genus Aspergillus, or a compound that is of benefit to animals or humans.

For example, the GOI could code for a pharmaceutically active protein or enzyme such as any one of the therapeutic compounds insulin, interferon, human serum albumin, human growth factor and blood clotting factors. In this regard, the transformed cell or organism could prepare acceptable quantities of the desired compound which would be easily retrievable from, the cell or organism. The GOI may even be a protein giving nutritional value to a food or crop. Typical examples include plant proteins that can inhibit the formation of anti-nutritive factors and plant proteins that have a more desirable amino acid composition (e.g. a higher lysine content than a non-transgenic plant). The GOI may even code for an enzyme that can be used in food processing such as chymosin, thaumatin and α-galactosidase. The GOI can be a gene encoding for any one of a pest toxin, an antisense transcript such as that for patatin or α-amylase, ADP-glucose pyrophosphorylase (e.g. see EP-A0455316), a protease antisense or a glucanase.

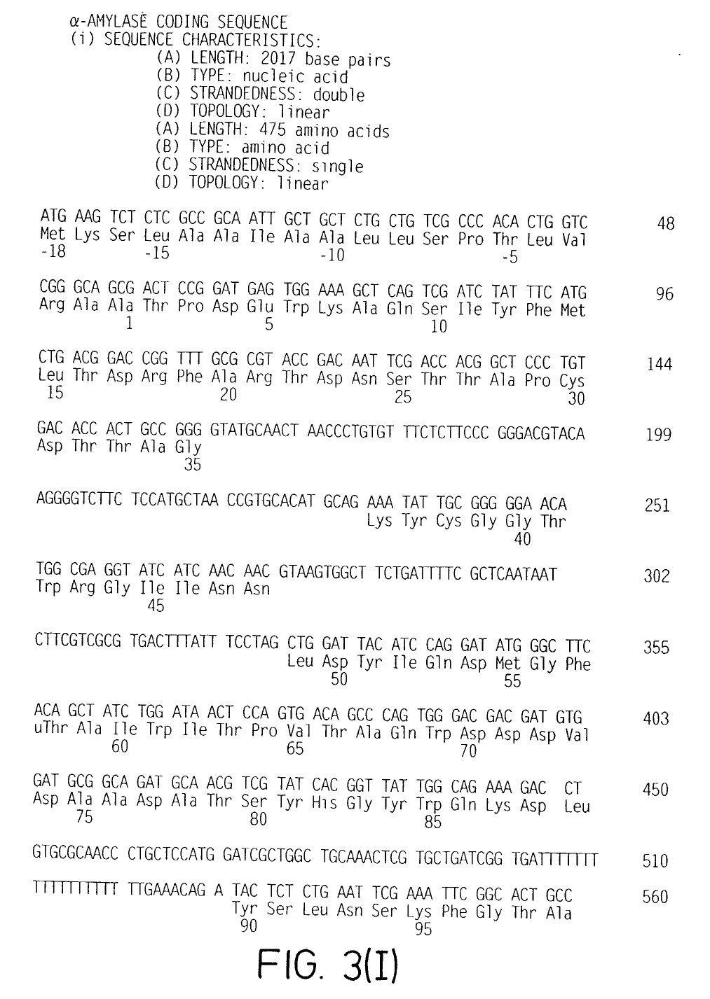

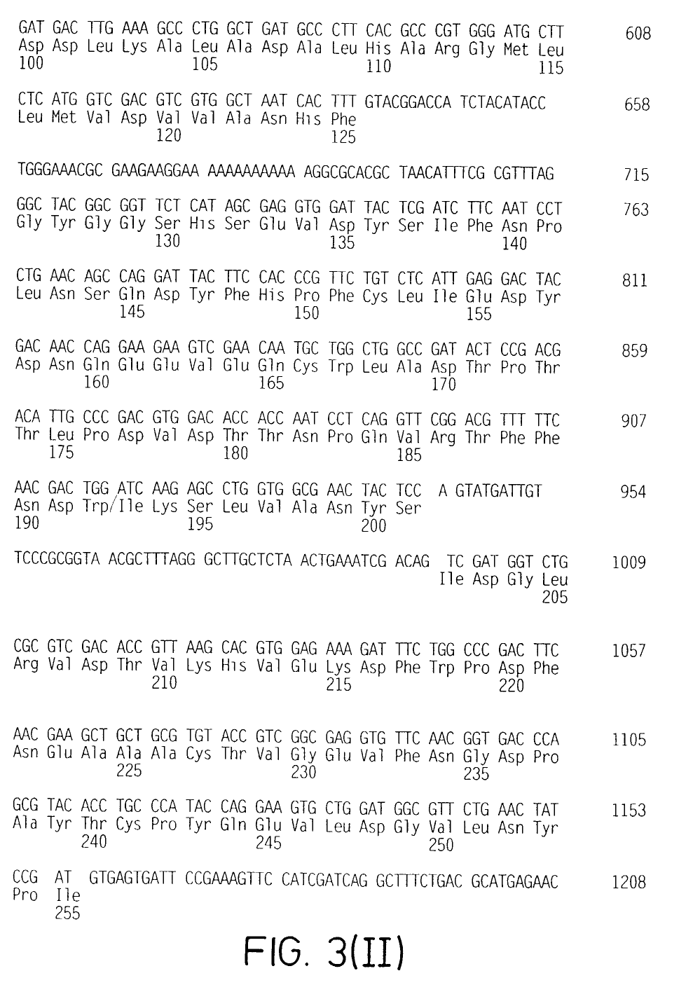

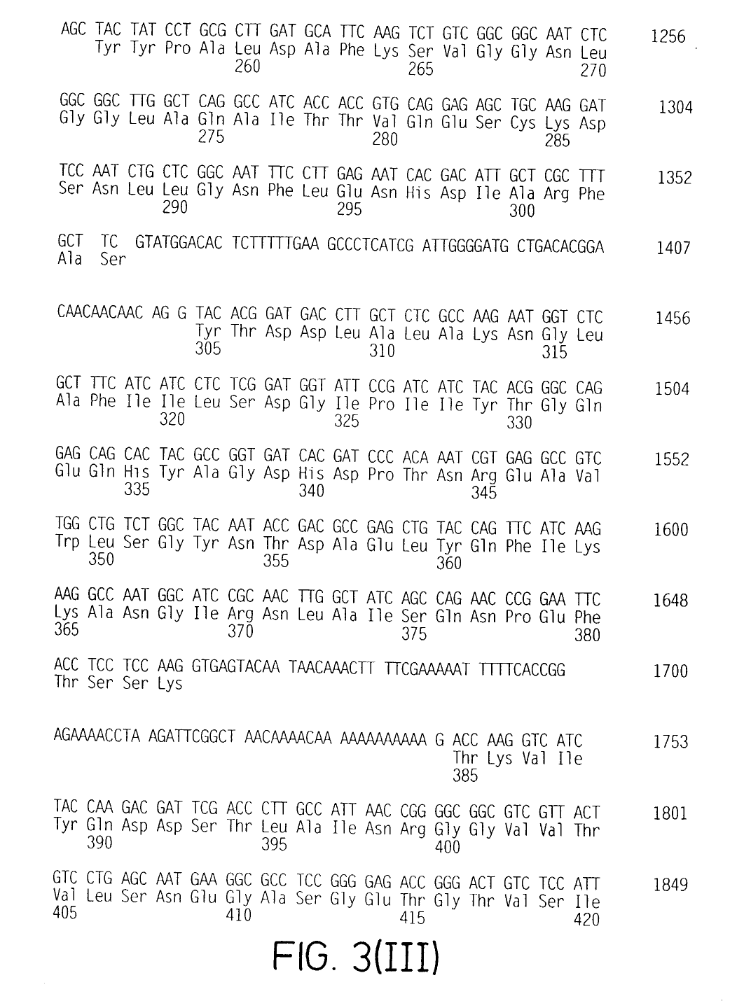

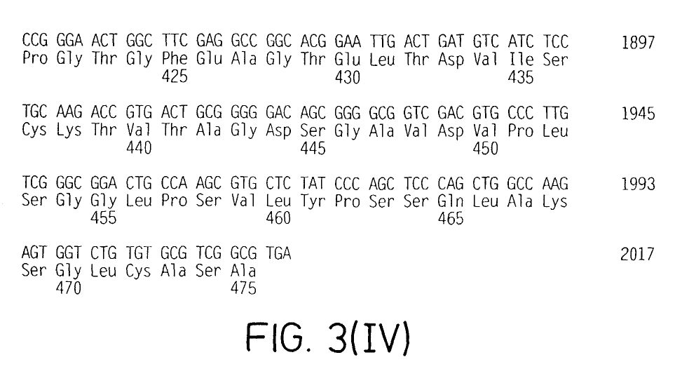

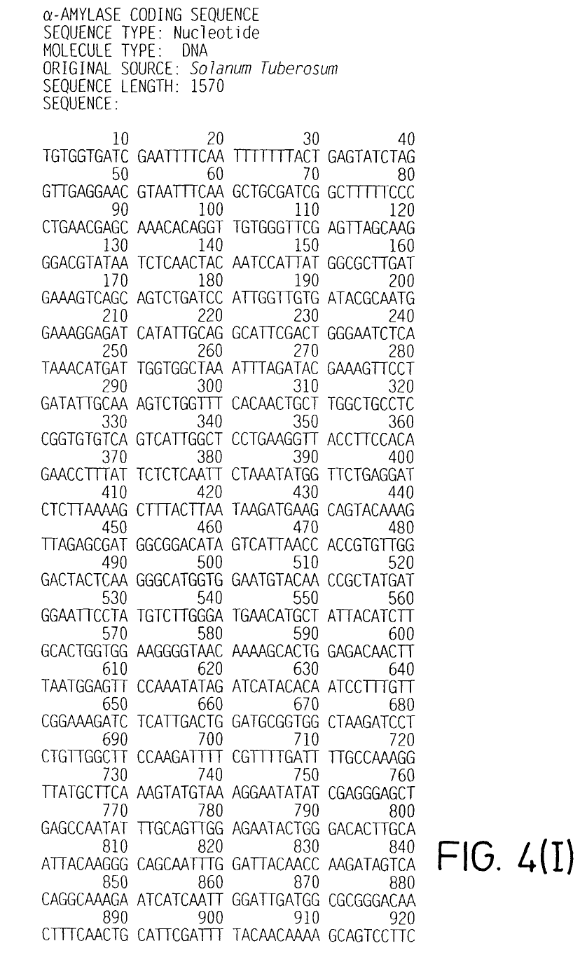

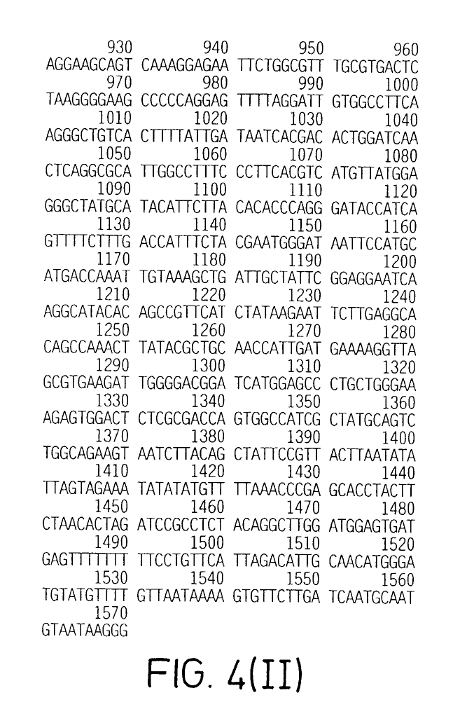

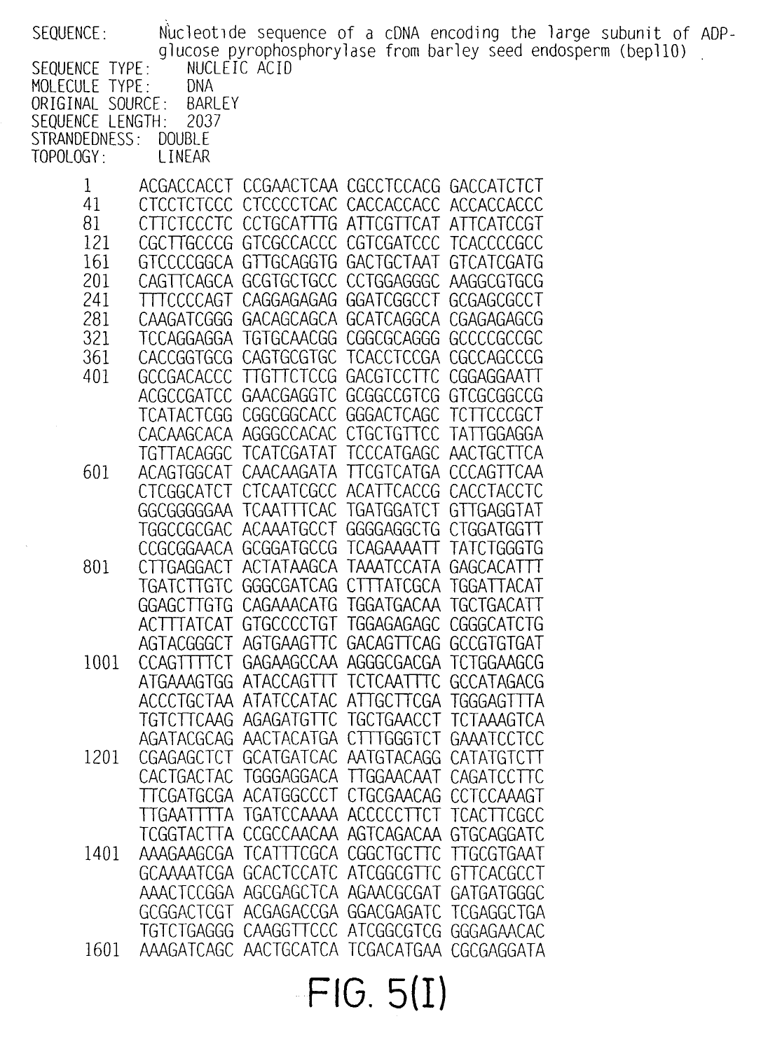

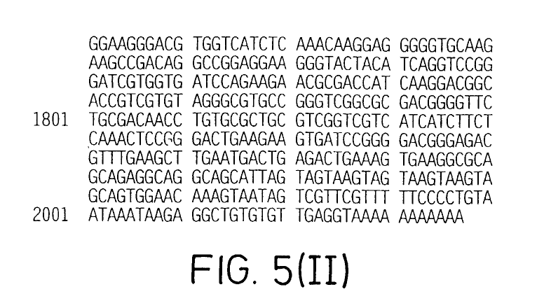

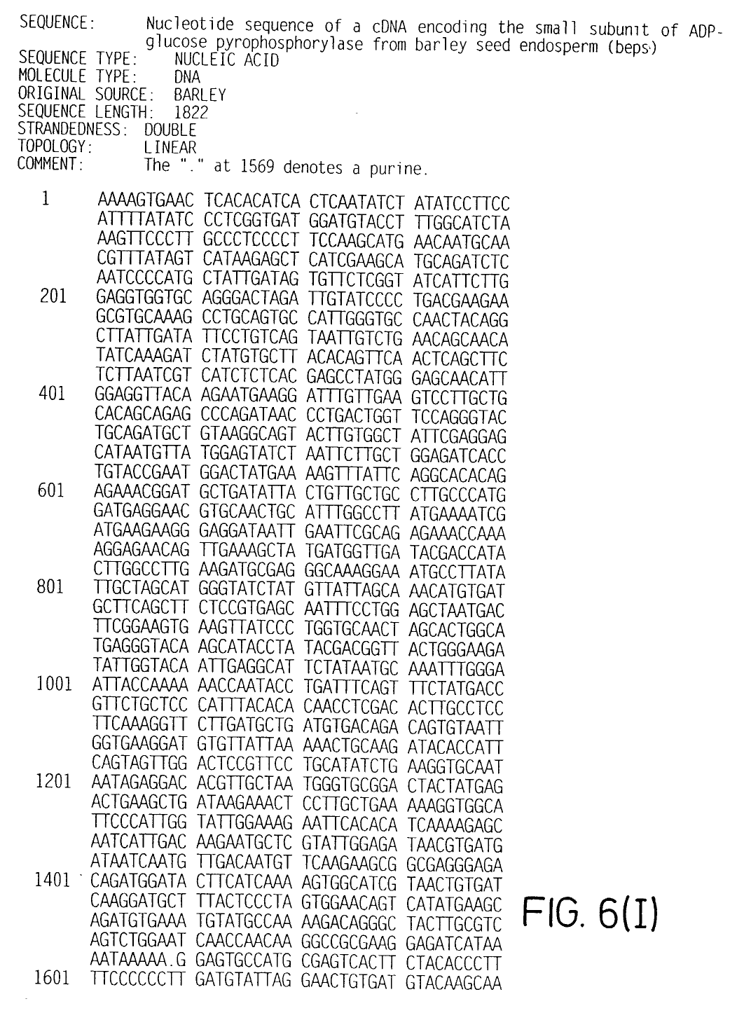

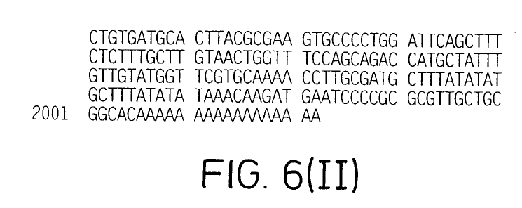

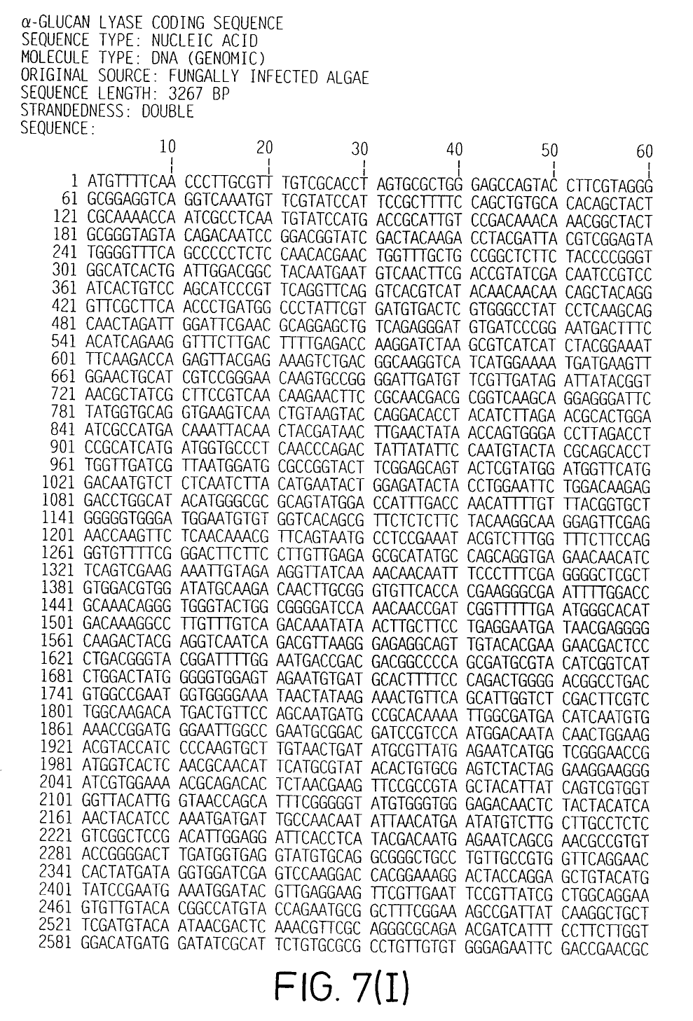

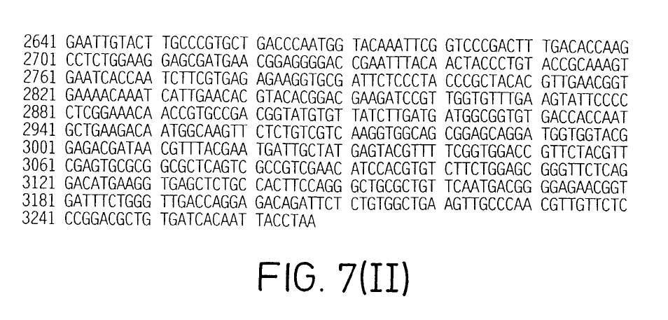

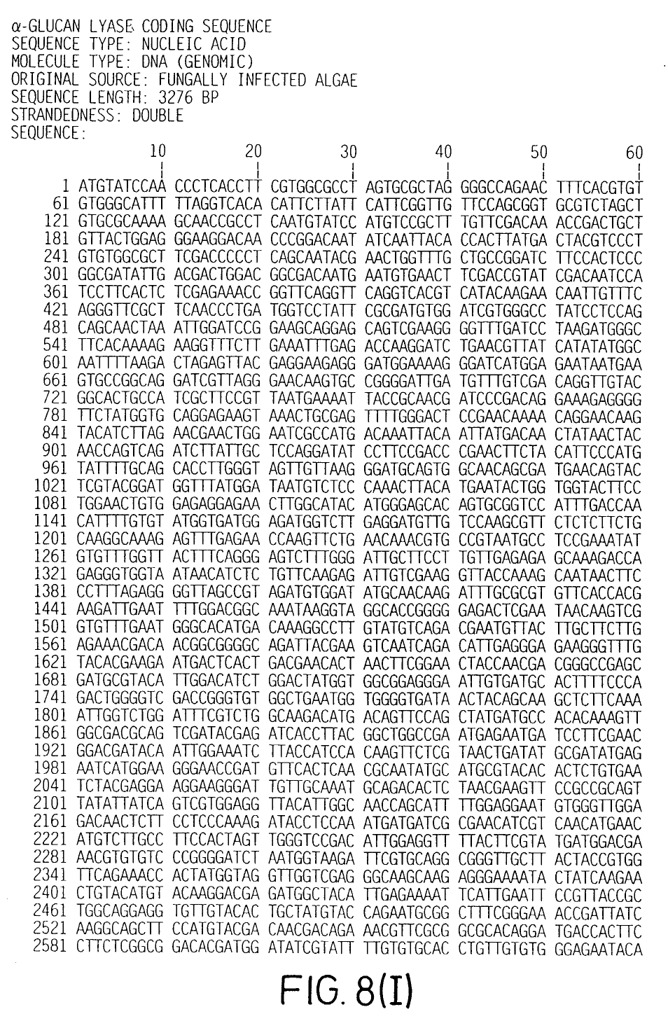

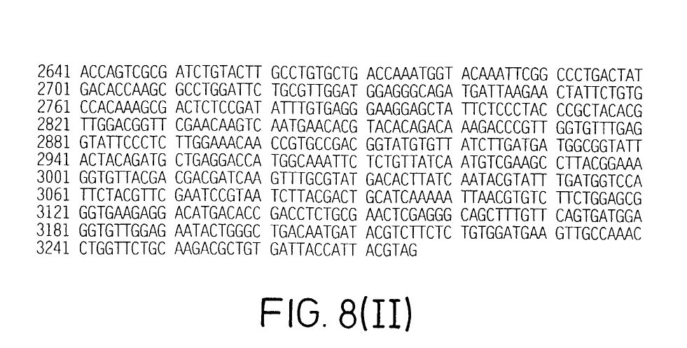

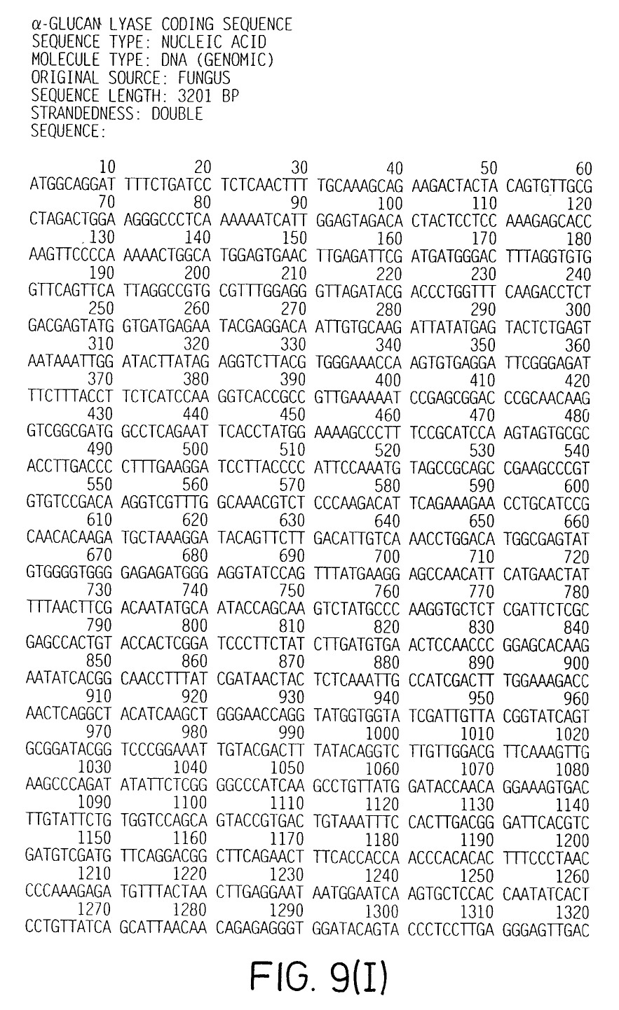

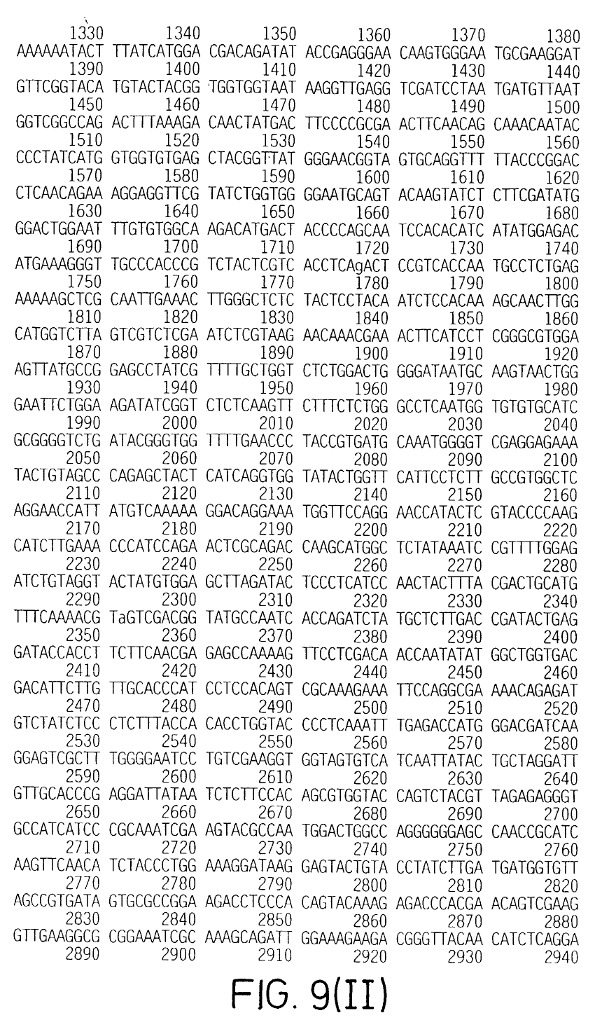

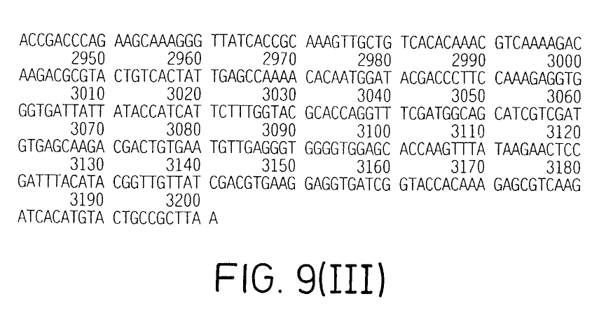

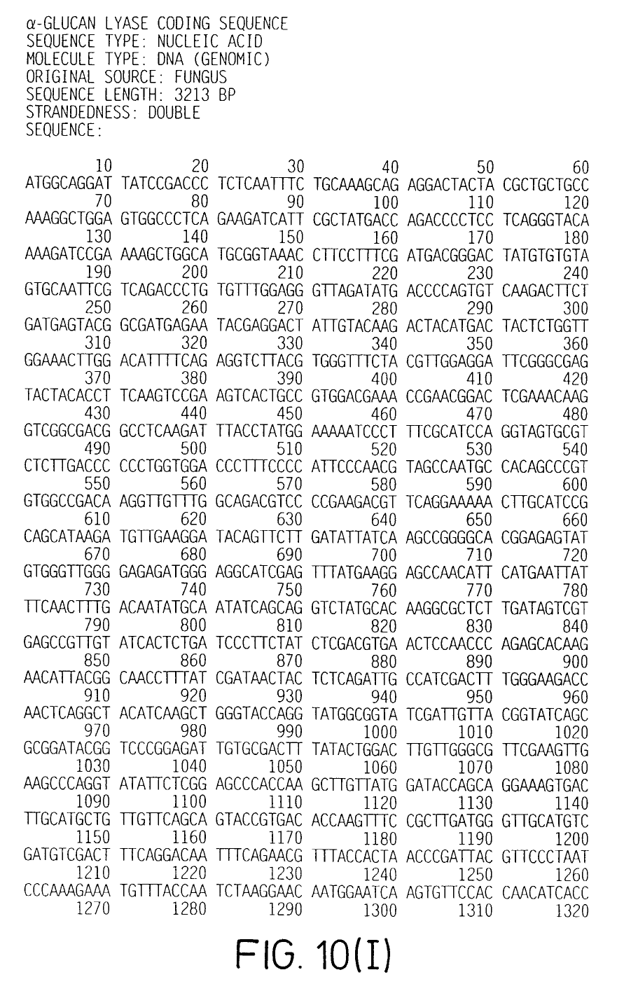

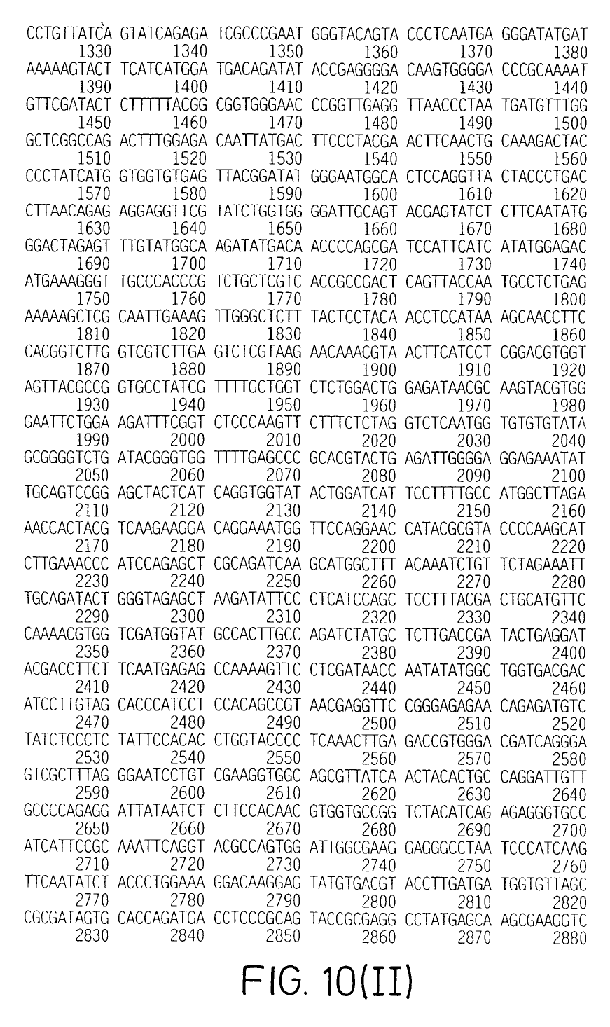

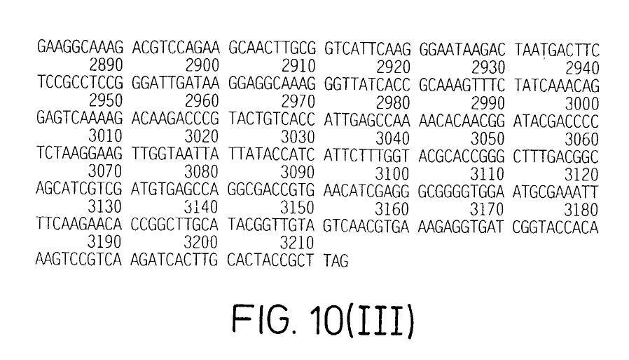

The GOI can be the nucleotide sequence coding for the α-amylase enzyme which is the subject of our co-pending UK patent application 9413439.2 filed on Jul 4, 1994, the sequence of which is shown in FIG. 3. The GOI can be the nucleotide sequence coding for the α-amylase enzyme which is the subject of our co-pending UK patent application 9421290.9 filed on Oct. 21, 1994, the sequence of which is shown in FIG. 4. The GOI can be any of the nucleotide sequences coding for the ADP-glucose pyrophosphorylase enzymes which are the subject of our co-pending PCT patent application PCT/EP94/01082 filed Apr. 7, 1994, the sequences of which are shown in FIGS. 5 and 6. The GOI can be any of the nucleotide sequences coding for the α-glucan lyase enzyme which are described in our co-pending PCT patent application PCT/EP94/03397 filed Oct. 15, 1994, the sequences of which are shown in FIGS. 7-10.

In one preferred embodiment, the GOI is a nucleotide sequence coding for the enzyme according to the present invention.

As mentioned above, a preferred host organism is of the genus Aspergillus, such as Aspergillus niger.

The transgenic Aspergillus according to the present invention can be prepared by following the teachings of Rambosek, J. and Leach, J. 1987 (Recombinant DNA in filamentous fungi: Progress and Prospects. CRC Crit. Rev. Biotechnol. 6:357-393), Davis R. W. 1994 (Heterologous gene expression and protein secretion in Aspergillus. In: Martinelli S. D., Kinghorn J. R. (Editors) Aspergillus: 50 years on. Progress in industrial microbiology vol 29. Elsevier Amsterdam 1994. pp 525-560). Ballance, D. J. 1991 (Transformation systems for Filamentous Fungi and an Overview of Fungal Gene structure. In: Leong. S. A., Berka R. M. (Editors) Molecular Industrial Mycology Systems and Applications for Filamentous Fungi. Marcel Dekker Inc. New York 1991. pp 1-29) and Turner G. 1994 (Vectors for genetic manipulation. In: Martinelli S. D., Kinghorn J. R. (Editors) Aspergillus: 50 years on. Progress in industrial microbiology vol 29, Elsevier Amsterdam 1994, pp. 641-666). However, the following commentary provides a summary of those teachings for producing transgenic Aspergillus according to the present invention.

Filamentous fungi have during almost a century been widely used in industry for production of organic compounds and enzymes. Traditional japanese koji and soy fermentations have used Aspergillus sp. for hundreds of years. In this century Aspergillus niger has been used for production of organic acids particular citric acid and for production of various enzymes for use in industry.

There are two major reasons for that filamentous fungi have been so widely used in industry. First filamentous fungi can produce high amounts of extracellular products, for example enzymes and organic compounds such as antibiotics or organic acids. Second filamentous fungi can grow on low cost substrates such as grains, bran, beet pulp etc. The same reasons have made filamentous fungi attractive organisms as hosts for heterologous expression according to the present invention.

In order to prepare the transgenic Aspergillus, expression constructs are prepared by inserting a GOI (such as an amylase or SEQ. I.D. No. 2) into a construct designed for expression in filamentous fungi.

Several types of constructs used for heterologous expression have been developed. The constructs contain the promoter according to the present invention (or if desired another promoter if the GOI codes for the enzyme according to the present invention) which is active in fungi. Examples of promoters other than that of the present invention include a fungal promoter for a highly expressed extracellulary enzyme, such as the glucoamylase promoter or the α-amylase promoter. The GOI can be fused to a signal sequence (such as that of the present invention or another suitable sequence) which directs the protein encoded by the GOI to be secreted. Usually a signal sequence of fungal origin is used, such as that of the present invention. A terminator active in fungi ends the expression system, such as that of the present invention.

Another type of expression system has been developed in fungi where the GOI is fused to a smaller or a larger part of a fungal gene encoding a stable protein. This can stabilize the protein encoded by the GOI. In such a system a cleavage site, recognized by a specific protease, can be introduced between the fungal protein and the protein encoded by the GOI, so the produced fusion protein can be cleaved at this position by the specific protease thus liberating the protein encoded by the GOI (“POI”). By way of example, one can introduce a site which is recognized by a KEX-2 like peptidase found in at least some Aspergilli. Such a fusion leads to cleavage in vivo resulting in protection of the POI and production of POI and not a larger fusion protein.

Heterologous expression in Aspergillus has been reported for several genes coding for bacterial, fungal, vertebrate and plant proteins. The proteins can be deposited intracellularly if the GOI is not fused to a signal sequence. Such proteins will accumulate in the cytoplasm and will usually not be glycosylated which can be an advantage for some bacterial proteins. If the GOI is equipped with a signal sequence the protein will accumulate extracellulary.

With regard to product stability and host strain modifications, some heterologous proteins are not very stable when they are secreted into the culture fluid of fungi. Most fungi produce several extracellular proteases which degrade heterologous proteins. To avoid this problem special fungal strains with reduced protease production have been used as host for heterologous production.

For the transformation of filamentous fungi, several transformation protocols have been developed for many filamentous fungi (Ballance 1991, ibid). Many of them are based on preparation of protoplasts and introduction of DNA into the protoplasts using PEG and Ca²⁺ ions. The transformed protoplasts then regenerate and the transformed fungi are selected using various selective markers Among the markers used for transformation are a number of auxotrophic markers such as argB, rypC, niaD and pyrG, antibiotic resistance markers such as benomyl resistance, hygromycin resistance and phleomycin resistance. A very common used transformation marker is the amdS gene of A. nidulans which in high copy number allows the fungus to grow with acrylamide as the sole nitrogen source.

Even though the enzyme, the nucleotide sequence coding for same and the promoter of the present invention are not disclosed in EP-B0470145 and CA-A-2006454, those two documents do provide some useful background commentary on the types of techniques that may be employed to put the present invention into practice. Some of these background teachings are now included in the following commentary.

The basic principle in the construction of genetically modified plants is to insert genetic information in the plant genome so as to obtain a stable maintenance of the inserted genetic material. Several techniques exist for inserting the genetic information, the two main principles being direct introduction of the genetic information and introduction of the genetic information by use of a vector system.

A review of the general techniques may be found in articles by Potrykus (Annu Rev Plant Physiol Plant Mol Biol [1991] 42:205-225) and Christou (Agro-Food-Industry Hi-Tech March/April 1994 17-27).

Thus, in one aspect, the present invention relates to a vector system which carries a promoter or nucleotide sequence or construct according to the present invention and which is capable of introducing the promoter or nucleotide sequence or construct into the genome of an organism, such as a plant.

The vector system may comprise one vector, but it can comprise two vectors. In the case of two vectors, the vector system is normally referred to as a binary vector system. Binary vector systems are described in further detail in Gynheun An et al. (1980). Binary Vectors. Plant Molecular Biology Manual A3, 1-19.

One extensively employed system for transformation of plant cells with a given promoter or nucleotide sequence or construct is based on the use of a Ti plasmid from Agrobacterium tumefaciens or a Ri plasmid from Agrobacterium rhizogenes An et al (1986), Plant Physiol. 81, 301-305 and Butcher D. N. et al. (1980), Tissue Culture Methods for Plant Pathologists, eds.: D. S. Ingrams and J. P. Helgeson, 203-208.

Several different Ti and Ri plasmids have been constructed which are suitable for the construction of the plant or plant cell constructs described above. A non-limiting example of such a Ti plasmid is pGV3850.

The promoter or nucleotide sequence or construct of the present invention should preferably be inserted into the Ti-plasmid between the terminal sequences of the T-DNA or adjacent a T-DNA sequence so as to avoid disruption of the sequences immediately surrounding the T-DNA borders, as at least one of these regions appear to be essential for insertion of modified T-DNA into the plant genome.

As will be understood from the above explanation, if the organism is a plant, then the vector system of the present invention is preferably one which contains the sequences necessary to infect the plant (e.g. the vir region) and at least one border part of a T-DNA sequence, the border part being located on the same vector as the genetic construct.

Furthermore, the vector system is preferably an Agrobacterium tumefaciens Ti-plasmid or an Agrobacterium rhizogenes Ri-plasmid or a derivative thereof, as these plasmids are well-known and widely employed in the construction of transgenic plants, many vector systems exist which are based on these plasmids or derivatives thereof.

In the construction of a transgenic plant the promoter or nucleotide sequence or construct of the present invention may be first constructed in a microorganism in which the vector can replicate and which is easy to manipulate before insertion into the plant. An example of a useful microorganism is E. coli, but other microorganisms having the above properties may be used. When a vector of a vector system as defined above has been constructed in E. coli, it is transferred, if necessary, into a suitable Agrobacterium strain, e.g. Agrobacterium tumefaciens. The Ti-plasmid harbouring the promoter or nucleotide sequence or construct of the invention is thus preferably transferred into a suitable Agrobacterium strain, e.g. A. tumefaciens, so as to obtain an Agrobacterium cell harbouring the promoter or nucleotide sequence or construct of the invention, which DNA is subsequently transferred into the plant cell to be modified.

As reported in CA-A-2006454, a large amount of cloning vectors are available which contain a replication system in E. coli and a marker which allows a selection of the transformed cells. The vectors contain for example pBR 322, pUC series, M13 mp series, pACYC 184 etc. In such a way, the nucleotide or construct or promoter of the present invention can be introduced into a suitable restriction position in the vector. The contained plasmid is used for the transformation in E. coli. The E. coli cells are cultivated in a suitable nutrient medium and then harvested and lysed. The plasmid is then recovered. As a method of analysis there is generally used sequence analysis, restriction analysis, elecrophoresis and further biochemical-molecular biological methods. After each manipulation, the used DNA sequence can be restricted and connected with the next DNA sequence. Each sequence can be cloned in the same or different plasmid. After each introduction method of the desired promoter or construct or nucleotide sequence according to the present invention in the plants the presence and/or insertion of further DNA sequences may be necessary. If, for example, for the transformation the Ti- or Ri-plasmid of the plant cells is used, at least the night boundary and often however the right and the left boundary of the Ti- and Ri-plasmid T-DNA, as flanking areas of the introduced genes, can be connected. The use of T-DNA for the transformation of plant cells has been intensively studied and is described in EP-A-120516; Hoekema, in: The Binary Plant Vector System Offset-drukkerij Kanters B. B., Alblasserdam, 1985, Chapter V; Fraley, et al., Crit. Rev. Plant Sci., 4:1-46; and An et al., EMBO J. (1985) 4:277-284.

Direct infection of plant tissues by Agrobacterium is a simple technique which has been widely employed and which is described in Butcher D. N. et al. (1980), Tissue Culture Methods for Plant Pathologists, eds.: D. S. Ingrams and J. P. Helgeson, 203-208. For further teachings on this topic see Potrykus (Annu Rev Plant Physiol Plant Mol Biol [1991] 42,205-225) and Christou (Agro-Food-Industry Hi-Tech March/April 1994 17-27). With this technique, infection of a plant may be done on a certain part or tissue of the plant, i.e. on a part of a leaf, a root, a stem or another part of the plant.

Typically, with direct infection of plant tissues by Agrobacterium carrying the promoter and/or the GOI, a plant to be infected is wounded, e.g. by cutting the plant with a razor or puncturing the plant with a needle or rubbing the plant with an abrasive. The wound is then inoculated with the Agrobacterium. The inoculated plant or plant part is then grown on a suitable culture medium and allowed to develop into mature plants.

When plant cells are constructed, these cells may be grown and maintained in accordance with well-known tissue culturing methods such as by culturing the cells in a suitable culture medium supplied with the necessary growth factors such as amino acids, plant hormones, vitamins, etc. Regeneration of the transformed cells into genetically modified plants may be accomplished using known methods for the regeneration of plants from cell or tissue cultures, for example by selecting transformed shoots using an antibiotic and by subculturing the shoots on a medium containing the appropriate nutrients, plant hormones, etc.

Further teachings on plant transformation may be found in EP-A-0449375.

In summation, the present invention provides a glucanase enzyme and the nucleotide sequence coding for the same. In addition, it provides a promoter that can control the expression of that, or another, nucleotide sequence. In addition it includes terminator and signal sequences for the same.

The following sample was deposited in accordance with the Budapest Treaty at the recognised depositary The National Collections of Industrial and Marine Bacteria Limited (NCIMB) at 23 St. Machar Drive, Aberdeen, Scotland, United Kingdom, AB2 1RY on Jan. 16, 1995:

E. coli containing plasmid pEGLA-3 { i.e. E. coli DH5α-pEGLA-3}. The deposit number is NCIMB 40704.

The present invention will now be described by way of example.

In the following Examples reference is made to the accompanying Figures in which

FIGS. 1-10 are sequences of promoters and GOIs of earlier patent applications that are useful for use with the aspects of the present invention;

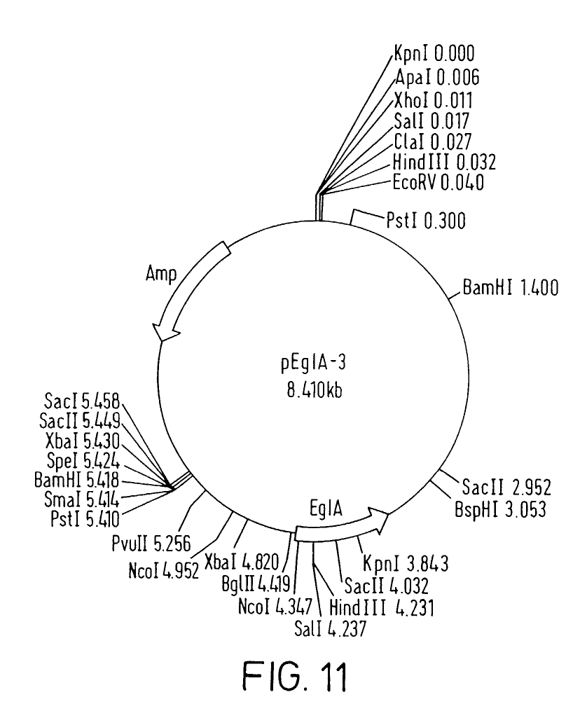

FIG. 11 is a plasmid map of plasmid pEGLA-3;

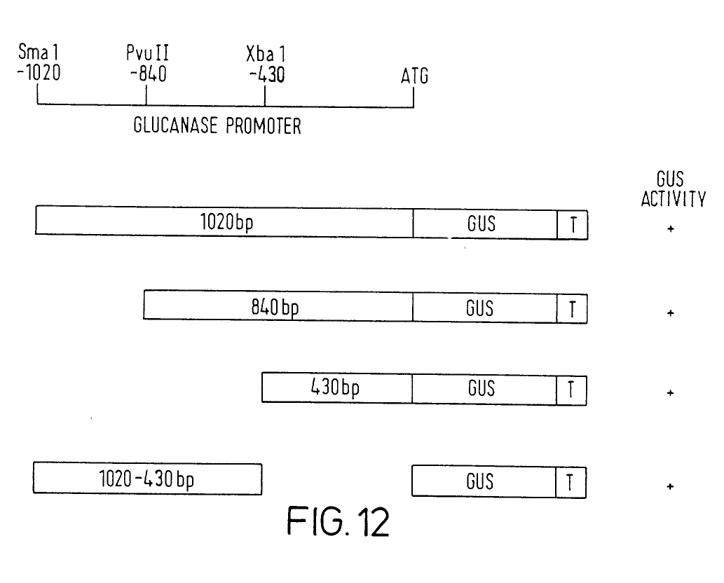

FIG. 12 is a schematic diagram of some promoter deletions;

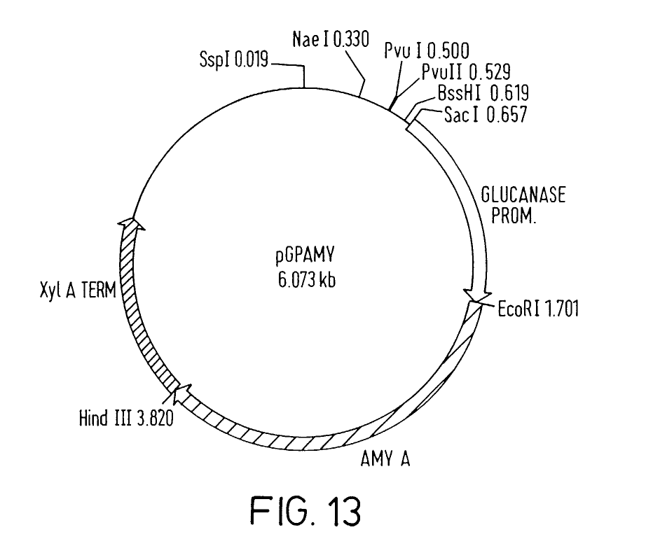

FIG. 13 is a plasmid map of pGPAMY;

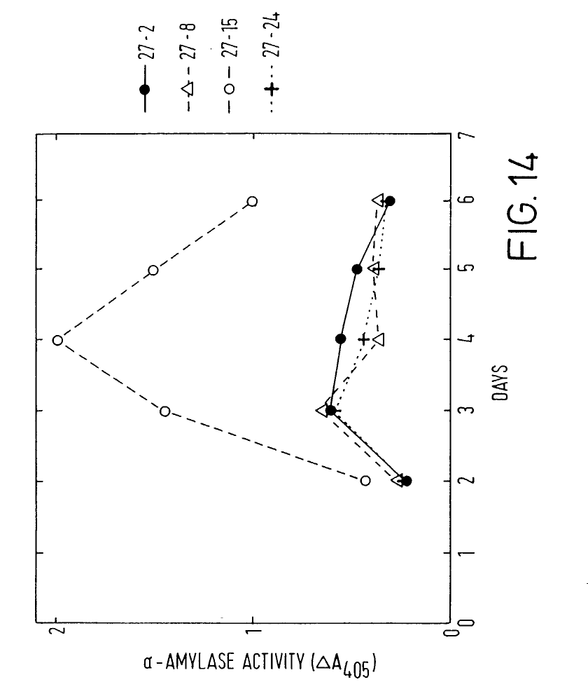

FIG. 14 is a graph;

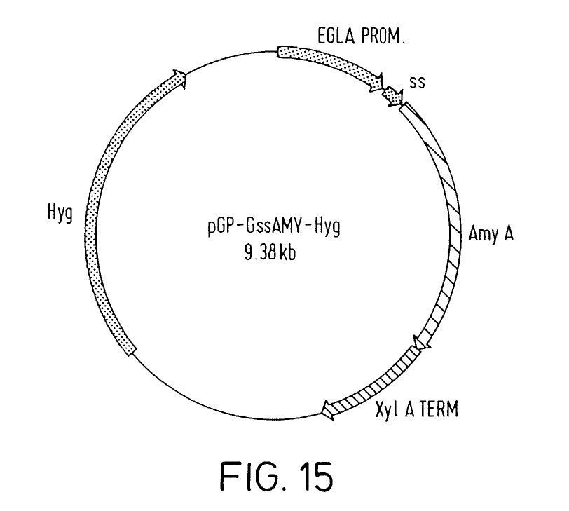

FIG. 15 is a plasmid map of pGP-GssAMY-Hyg;

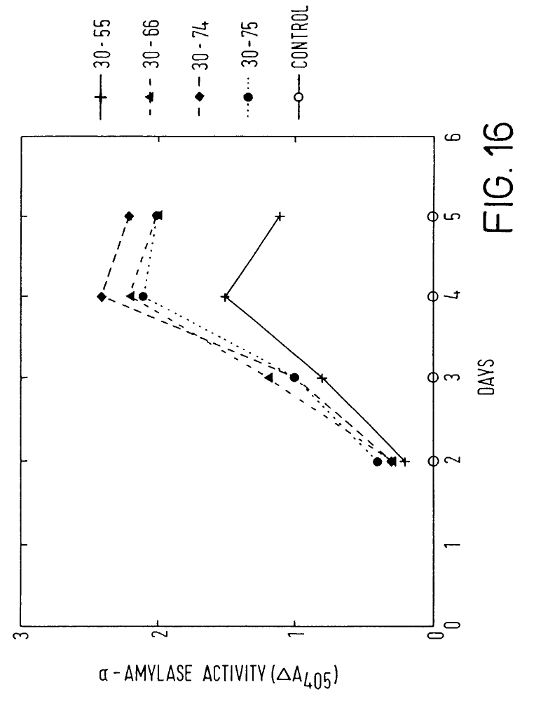

FIG. 16 is a graph; and

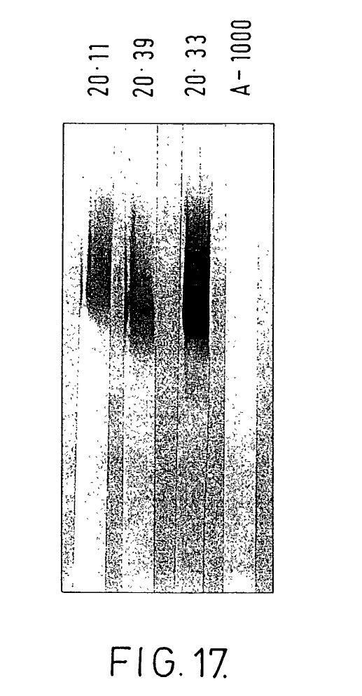

FIG. 17 is a Western Blot.

The following Examples discuss recombinant DNA techniques. General teachings of recombinant DNA techniques may be found in Sambrook, J., Fritsch, E. F., Maniatis T. (Editors) Molecular Cloning. A laboratory manual. Second edition. Cold Spring Harbour Laboratory Press. New York 1989.

Purification of the β-Glucanase

Aspergillus niger 3M43 was grown in medium containing wheat bran and beet pulp. The fermentation broth was separated from the solid part of the broth by filtration Concentrated fermentation broth was then loaded on a 25×100 mm Q-SEPHAROSE (Pharmacia) high Performance column, equilibrated with 20 mM Tris, HCl pH 7.5, and a linear gradient from 0-500 mM NaCl was performed and fractions of the eluate was collected. The β-glucanase eluted at ca 100 mM NaCl. The fractions containing glucanase were combined and desalted using a 50×200 mm G-25 SEPHAROSE Superfine (Pharmacia). The column was then eluted with distilled water. After desalting the enzyme was concentrated using High-Trap spin columns.

Next the concentrated and desalted fractions were subjected to gel filtration on a 50×600 mm SUPERDEX 50 column. The sample was loaded and the column was eluted with 0.2 M Phosphate buffer pH 7.0 plus 0.2 M NaCl, and fractions of the eluate were collected. The fractions containing glucanase were combined and desalted and concentrated as described above.

The combined fractions were loaded on a 16×100 mm PhenylSEPHAROSE High Performance column (Pharmacia), equilibrated with 50 mM Phosphate buffer pH 6.0, containing 1.5 M (NH₄)₂SO₄. A gradient where the (NH₄)₂SO₄ concentration was varied from 1.5-0 M was applied and the eluate collected in fractions. The fractions containing glucanase were combined. The purity of the β-1,4-glucanase was evaluated SDS-PAGE using the Phast system gel (Pharmacia).

Characterization

The molecular weight of the purified glucanase was determined by mass spectrometry using laser desorption technology. The MW of the glucanase was found to be 24,235 D±50 D.

The pI value was determined by use of a Broad pI Kit (Pharmacia). The glucanase has a pI value of about 4.

After SDS-PAGE analysis treatment PAS reagent showed that the glucanase is not glycosylated. The PAS staining was done according to the procedure of I. Van-Seuninsen and M. Davril (1992) Electrophoresis 13 pp 97-99.

Amino Acid Sequencing of the β-Glucanase

The enzyme was digested with endoproteinase Lys-C sequencing grade from Boehringer Mannheim using a modification of the method described by Stone & Williams 1993 (Stone, K. L. and Williams, K. R. (1993). Enzymatic digestion of Proteins and HPLC Peptide Isolation. In : Matsudaira P. (Editor). A practical Guide to Protein and Peptide Purification for Microsequencing. Second Edition. Academic Press, San Diego 1993. pp 45-73).

Freeze dried β-glucanase (0.4 mg) was dissolved in 50 μl of 8M urea, 0.4 M NH₄HCO₃, pH 8.4. After overlay with N₂ and addition of 5 μl of 45 mM DTT, the protein was denatured and reduced for 15 min at 50° C. under N₂. After cooling to RT, 5 μl of 100 mM iodoacetamide was added for the cysteines to be derivatised for 15 min at RT in the dark under N₂. Subsequently, 90 μl of water and 5 μg of endoproteinase Lys-C in 50 μl of 50 mM Tricine and 10 mM EDTA, pH 8.0, was added and the digestion was carried out for 24 h at 37° C. under N₂. The resulting peptides were separated by reversed phase HPLC on a VYDAC C18 column (0.46×15 cm; 10 μm; The Separations Group; California) using solvent A: 0.1% TFA in water and solvent B: 0.1% TFA in acetonitrile. Selected peptides were rechromatographed on a Develosil C18 column (0.46×10 cm; 3 μm) using the same solvent system prior to sequencing on an Applied Biosystems 476A sequencer using pulsed-liquid fast cycles.

The following peptide sequences were found:

SEQ I.D. No. 4

SEQ I.D. No. 5

SEQ I.D. No. 6

SEQ I.D. No. 7

SEQ I.D. No. 8

Isolation of a PCR Clone of a Fragment of the Gene

PCR primers were synthesised using an Applied Biosystems DNA synthesiser model 392. In this regard, PCR primers were synthesized from two of the found peptide sequences, WEVWYGT from Seq I.D. No. 4 and WTWSGG from Seq I.D. No. 7. The primer derived from WEVWYGT (reversed) is shown as Seq I.D. No. 9 and the primer derived from WTWSGG is shown as Seq I.D. No. 10—see below:

| | |
|---|---|
| TGG ACN TGG WSN GGN GG | SEQ. I.D. No. 10 |
| 17 mer 256 mixture | |
| CTN CCR TAC CAN ACY TCC CA | SEQ. I.D. No. 9 |
| 20 mer 64 mixture | |

PCR amplification was performed with 100 pmol of each of these primers in 100 μl reactions using the Amplitaq II kit (Perkin Elmer). The program was:

| STEP | TEMP | TIME |
|---|---|---|
| 1 | 94° C. | 2 min |
| 2 | 94° C. | 1 min |
| 3 | 55° C. | 2 min |
| 4 | 72° C. | 2 min |
| 5 | 72° C. | 5 min |
| 6 | 5° C. | SOAK |

Steps 2-4 were repeated for 40 cycles.

The program was run on a PERKIN ELMER DNA Thermal Cycler.

A 350 bp amplified fragment was isolated and cloned into a pT7-Blue T-vector according to the manufacturer's instructions (Novagen). A fragment was isolated and sequenced. The found sequence showed that it was indeed a part of the glucanase gene.

Isolation of A. niger Genomic DNA

1 g. of frozen A. niger mycelium was ground in a mortar under liquid nitrogen. Following evaporation of the nitrogen cover, the ground mycelium was extracted with 15 ml of an extraction buffer (100 mM Tris HCl, pH 8.0, 0.50 mM EDTA, 500 mM NaCl, 10 mM β-mercaptoethanol) containing 1 ml 20% sodium dodecyl sulphate. After incubation at 65° C. for 10 min, 5 ml 5 M KAc, pH 5.0, was added and the mixture further incubated, after mixing, on ice for 20 mins. The mixture was then centrifuged for 20 mins. and the supernatant mixed with 0.6 vol. isopropanol to precipitate the extracted DNA. After further centrifugation for 15 mins, the DNA pellet was dissolved in 0.7 ml TE (10 mM Tris, HCl pH 8.0, 1 mM EDTA) and precipitated with 75 μl 3M NaAc, pH 4.8, and 500 μl isopropanol. After centrifugation the pellet was washed with 70% ETOH and dried under vacuum. The DNA was dissolved in 200 μl TE and stored at −20° C.

Construction of a Library

20 μg genomic DNA was partly digested with Tsp509I, which gives ends which are compatible with EcoRI ends. The digested DNA was separated on a 1% agarose gel and fragments of 4-10 kb was purified. A λZAPII EcoRI/CIAP kit from Stratazene was used for library construction according to the manufacturers instructions. 2 μl of the ligation (totally 5 μl) was packed with Gigapack Gold II packing extract according to the manufacturer's instructions (Stratagene). The library contained 650,000 independent clones.

Screening of the Library

2×50,000 pfu was plated on NZY plates (5 g NaCl, 2 mg MgSO₄, 7H₂O, 5 g yeast extract, 10 g casein hydrolysate, 15 g agar per liter) and plaquelifts were done on Hybond N sheets (Amersham). The sheets were hybridized with the PCR clone labelled with ³²P dCTP (Amersham) using Ready-to-go labelling kit from Pharmacia. The plaquelifts and hybridization were done in duplicate and positive clones were reckoned only when hybridization could be detected on both sheets. The nucleotide sequence of the present invention was sequenced using a ALF-laser fluorescence sequencer (Pharmacia). The sequence contained all the found amino acid sequence, confirming that the isolated gene indeed encoded the β-1,4-endoglucanase.

Sequence Information

SEQ. ID. No. 12 presents the promoter sequence, the enzyme coding sequence, the terminator sequence and the signal sequence and the amino acid sequence of the enzyme of the present invention.

Testing Enzyme Activity

The purified protein was assayed for endo β-1,4 glucanase activity using Azurine-crosslinked barley β-glucan tablet (trade name: Glucazyme tablets supplied by Megazyme, Australia) by the instructions given by the manufacturer.

The purified enzyme gave a high activity on this substrate. Typically the enzyme has a specific activity of 2250 micromol glucose per min per mg of protein.

Induction of the Eg1A Gene: Identification of Inducing Carbon Source

The Table below shows the identification of a number of high and low molecular weight inducers of the glucanase promoter. This analysis was carried out using the full length glucanase promoter of the present invention fused to the E. coli β-2glucuronidase gene.

The inducing strength of different carbon sources was determined quantitatively by measuring the intracellular GUS specific activity to hydrolyse p-nitrophenol glucuronide.

| CARBON SOURCE | GUS ACTIVITY |
|---|---|
| (1%) | (units/mg)-24 hours |

| | |
|---|---|
| xylose | 12.91 |
| xylitol | 10.62 |
| arabinose | 8.50 |
| arabitol | 14.40 |
| glucose | 20.25 |
| cellubiose | 19.44 |
| xylo-oligomer 70 | 11.80 |
| glucopyranoside | 19.70 |
| methyl-xylopyranoside | 12.60 |
| xyloglucan | 13.90 |
| pectin | 9.70 |
| arabinogalactan | 30.20 |
| arabitol + glucose | 29.50 |

Surprisingly glucose, which is normally a potent catabolite repressor, induces the glucanase promoter.

Accordingly, the present invention also relates to a promoter that is inducible by glucose.

In addition, the present invention relates to the use of glucose to induce a promoter.

These aspects of the present invention are different to the teachings of WO 94/04673 which discloses a fungal promoter that is active in the presence of glucose. In this regard, the promoter of the present invention is not only active in the presence of glucose but that it is also inducible by glucose

One of the advantages of having a glucanase promoter that is inducible by glucose is that the promoter can be used to express a GOI, and thereby be used to prepare a POI (such as an heterologous POI), in a glucose containing environment. This is important because glucose is one of preferred carbon sources for biomass accumulation. In addition, glucose containing media are expected to produce lower amounts of proteases thereby providing better yields of the POI. In addition, the use of a derepressed promoter in a derepressed host strain will increase the speed and efficiency of reaction media, such as a fermentation reaction medium. In addition, the use of mixed carbon sources during fermentation will allow the efficient induction of this promoter, for example at low levels of glucose and a cheap carbon source (e.g. sugar beet pulp).

Effects of Promoter Deletions on the Regulation of the Expression of the Glucanase Gene

A series of deletion studies, which are shown in FIG. 12, were performed. In these studies, the different promoter deletion constructs shown in FIG. 12 were fused to the GUS gene. The activity of the reporter gene was assayed qualitatively. The results showed that none of the deletions abolished the inducibility of the glucanase promoter. These results indicate the presence of multiple sites for transcriptional activation and initiation of transcription.

### Heterologous Protein Production Using Transformants of Aspergillus niger Comprising the Glucanase Promoter (Gp) and the Glucanase Signal Seourence (Gss)

Transformation of Aspergillus niger

The protocol for transformation of A. niger was based on the teachings of Buxton, F. P., Gwynne, D. I., Davis, R. W. 1985 (Transformation of Aspergillus niger using the argB gene of Aspergillus nidulans. Gene 37, 07-214). Daboussi, M. J., Djeballi, A., Gerlinger, C., Blaiseau, P. L., Cassan, M., Lebrun, M. H., Parisot, D., Brygoo, Y. 1989 (Transformation of seven species of filamentous fungi using the nitrate reductase gene of Aspergillus nidulans. Curr. Genet. 15:453-456) and Punt, P. J., van den Hondel, C. A. M. J. J. 1992 (Transformation of filamentous fungi based on hygromycin B and Phleomycin resistance markers. Meth. Enzym. 216:447-457).

For the purification of protoplasts, spores from one PDA (Potato Dextrose Agar—from Difco Lab, Detroit) plate of fresh sporulated N400 (CBS 120.49, Centraalbureau voor Schimmelcultures, Baarn) (7 days old) are washed off in 5-10 ml water. A shake flask with 200 ml Potato Dextrose Broth (difco 0549-17-9, Difco Lab, Detroit) is inoculated with this spore suspension and shaken (250 rpm) for 16-20 hours at 30° C.

The mycelium is harvested using Miracloth paper and 3-4 g wet mycelium are transferred to a sterile petri dish with 10 ml STC (1.2 M sorbitol, 10 mM Tris HCl pH 7.5, 50 mM CaCl₂) with 75 mg lysine enzymes (Sigma L-2265) and 4500 units lyticase (Sigma L-8012).

The mycelium is incubated with the enzyme until the mycelium is degraded and the protoplasts are released. The degraded mycelium is then filtered through a sterile 60 μm mesh filter. The protoplasts are harvested by centrifugation 10 min at 2000 rpm in a swing out rotor. The supernatant is discarded and the pellet is dissolved in 8 ml 1.5 M MgSO₄ and then centrifuged at 3000 rpm for 10 min.

The upper band, containing the protoplasts is transferred to another tube, using a transfer pipette and 2 ml 0.6 M KCl is added. Carefully 5 ml 30% sucrose is added on the top and the tube is centrifuged 15 min at 3000 rpm.

The protoplasts, lying in the interface band, are transferred to a new tube and diluted with 1 vol. STC. The solution is centrifuged 10 mm at 3000 rpm. The pellet is washed twice with STC, and finally solubilized in 1 ml STC. The protoplasts are counted and eventually concentrated before transformation.

For the transformation, 100 μl protoplast solution (10⁶-10⁷ protoplasts) are mixed with 10 μl DNA solution containing 5-10 μg DNA and incubated 25 min at room temperature. Then 60% PEG-4000 is carefully added in portions of 200 μl, 200 μl and 800 μl. The mixture is incubated 20 min at room temperature. 3 ml STC is added to the mixture and carefully mixed. The mixture is centrifugated 3000 rpm for 10 min.

The supernatant is removed and the protoplasts are solubilized in the remaining of the supernatant, 3-5 ml topagarose is added and the protoplasts are quickly spread on selective plates.

Glucanase Promoter and Heterologous Gene Expression

FIG. 13 shows the expression vector pGPAmy that was used in these studies. This expression vector comprises the glucanase promoter fused to the Thermomyces lanuginosus precursor form of the α-amylase gene. Transcription from the promoter is terminated using the xylanase A terminator. This construct was used in a co-transformation experiment with the hygromycin resistance gene as the selectable marker.

The production of α-amylase using four independent transformants containing the expression vector pGPAmy when grown on sugar beet pulp and wheat bran is shown in FIG. 14. The α-amylase activity was first detected in the culture medium after 48 hours of growth. A peak of enzyme activity was observed after days 3 and 4.

Glucanase Signal Sequence & Heterologous Protein Production

For these studies, the expression vector pGPGssAmyHyg was used.

The vector pGPGssAmyHyg is shown in FIG. 15. This vector comprises the mature α-amylase gene translationally fused to the glucanase signal peptide (labelled ss). In addition, this vector comprises the promoter of the present invention (labelled EG1.A) and the xylanase A terminator. Transcription from this vector is therefore under the control of the glucanase promoter and termination by the xylanase A terminator.

This construct was used to test inter alia the efficiency of the signal peptide in heterologous protein secretion.

FIG. 16 shows the results of the induction of α-amylase by use of the construct in strain 6M179 when grown in sugar beet pulp/wheat bran. The results show that the enzyme activity was localised in the culture medium and was first detected after 48 hours of growth. Accumulation of enzyme activity was observed at day 4.

Western Blot

FIG. 17 shows a Western blot of proteins from the supernatant of three independent transformants separated by SDS-PAGE and blotted to a membrane. A synthetic peptide with 15 amino acid residues of T. lanuginosus α-amylase recognised a single band on Western blots of culture supernatants from the transformants.

Antibody Production

Antibodies were raised against the enzyme of the present invention by injecting rabbits with the purified enzyme and isolating the immunoglobulins from antiserum according to procedures described according to N Harboe and A Ingild (“Immunization, Isolation of Immunoglobulins, Estimation of Antibody Titre” In A Manual of Quantitative Immunoelectrophoresis, Methods and Applications, N H Axelsen, et al (eds.), Universitetsforlaget, Oslo, 1973) and by T G Cooper (“The Tools of Biochemistry”, John Wiley & Sons, New York, 1977).

### Summary

Even though it is known that Aspergillus niger produces several enzymes which can degrade β-glucan, the present invention provides a novel and inventive β- 1,4-endoglucanase, as well as the coding sequence therefor, the termination sequence therefor, the signal sequence therefor, and the promoter for those sequences An important advantage of the present invention is that the enzyme can be produced in high amounts. In addition, the promoter and the regulatory sequences (such as the signal sequence and the terminator) can be used to express or can be used in the expression of GOIs in organisms, such as in A. niger.

The enzyme of the present invention is advantageous for feed supplements. In addition, it can be used in the brewing industry as it has a high fibre-conversion potential. In addition, there are fewer processing problems when the enzyme is used, particularly with non-starchy polysaccharides. In addition, the enzyme efficiently degrades β-glucans, therefore it can be used advantageously in the brewing industry to lower viscosity and also improve the filterability of beer. This is important as large molecular weight glucans in beer and the like can cause filtration difficulties and give rise to sediments, gels and hazes.

The signal sequence of the present invention is useful for secretion of a POI, such as a heterologous POI, thereby improving the quality and quantity of the POI.

The present invention envisions an isolated glucanase enzyme, e.g., having the sequence shown as SEQ ID NO: 1, or homology thereto or a fragment thereof, for instance as herein discussed, e.g., a glucanase enzymatically active fragment of SEQ ID NO: 1 and/or polypeptide having homology to SEQ ID NO: 1 (with homology and fragment as herein discussed with homology and fragment as herein discussed, e.g., at least 75%, preferably at least 85%, more preferably at least 90%, advantageously at least 95%, and more advantageously at least 98% homology; and, the polypeptide advantageously has glucanase enzymatic activity). The present invention also comprehends an isolated glucanase enzyme coded for by a nucleic acid molecule having the nucleotide sequence shown as SEQ ID NO: 2 or homology thereto or a fragment thereof, for instance, as herein discussed, as well as an isolated nucleic acid molecule having the sequence shown as SEQ ID NO: 2 or homology thereto or a fragment thereof, for instance as herein discussed. For instance, such a nucleotide sequence encoding a glucanase enzymatically active fragment of SEQ ID NO: 1 and/or polypeptide having homology to SEQ ID NO: 1 (with homology and fragment as herein discussed, e.g., at least 75%, preferably at least 85%, more preferably at least 90%, advantageously at least 95%, and more advantageously at least 98% homology; and, the polypeptide advantageously has glucanase enzymatic activity). The present invention additionally provides an isolated nucleic acid molecule coding for the glucanase enzyme or a glucanase enzymatically active fragment thereof, e.g., of SEQ ID NO: 1 and/or polypeptide having homology to SEQ ID NO: 1 (with homology and fragment as herein discussed, e.g., at least 75%, preferably at least 85%, more preferably at least 90%, advantageously at least 95%, and more advantageously at least 98% homology; and, the polypeptide advantageously has glucanase enzymatic activity).

The present invention further provides a method of degrading a glucan polymer comprising adding to the glucan polymer the glucanase enzyme or a glucanase enzymatically active fragment thereof, e.g., of SEQ ID NO: 1 and/or polypeptide having homology to SEQ ID NO: 1 (with homology and fragment as herein discussed, e.g., at least 75%, preferably at least 85%, more preferably at least 90%, advantageously at least 95%, and more advantageously at least 98% homology; and, the polypeptide advantageously has glucanase enzymatic activity). The method can further comprise determining the extent of glucan polymer degradation. The invention further involves a method of degrading a glucan polymer comprising adding to the glucan polymer a glucanase enzyme having the sequence shown as SEQ ID NO: 1 or a homologue or variant thereof having at least 75% homology thereto and glucanase enzymatic activity such as the glucanase activity of the enzyme shown as SEQ ID NO: 1. This method too can further comprise determining the extent of polymer degradation. Advantageously, the glucanase enzyme has the sequence shown as SEQ ID NO: 1. The invention further comprehends a method for degrading a glucan polymer comprising adding to the glucan polymer a glucanase enzyme, wherein the enzyme is coded for by a first nucleic acid molecule that hybridizes to a second nucleic acid molecule, and the second nucleic acid molecule has the nucleotide sequence shown as SEQ ID NO: 2, and the glucanase enzyme has glucanase enzymatic activity of the glucanase enzyme shown as SEQ ID NO: 1. And, this method too can additionally include determining the extent of glucan degradation.

The invention further involves each inventive isolated nucleic acid molecule (e.g., a nucleic acid molecule of SEQ ID NO: 2 or a fragment thereof or a nucleic acid molecule having homology thereto, such as herein discussed; for instance, having at least 75%, preferably at least 85%, more preferably at least 90%, advantageously at least 95%, and more advantageously at least 98% homology and/or encoding a glucanase enzymatically active polypeptide) operatively linked to a promoter, as well as a construct or vector or plasmid comprising or expressing each inventive isolated nucleic acid molecule. Even further, the invention comprehends a process for preparing a glucanase enzyme, e.g., having a nucleotide sequence of SEQ ID NO: 1, or a glucanase enzymatically active fragment thereof, e.g., of SEQ ID NO: 1, and/or polypeptide having homology to SEQ ID NO: 1 (with homology and fragment as herein discussed, e.g., at least 75%, preferably at least 85%, more preferably at least 90%, advantageously at least 95%, and more advantageously at least 98% homology and the polypeptide advantageously having glucanase enzymatic activity). The process can comprise expressing an inventive nucleic acid molecule, e.g., such a nucleic acid molecule having the nucleotide sequence shown as SEQ ID NO: 2 or having homology thereto and/or a fragment thereof (such as herein discussed, e.g., at least 75%, preferably at least 85%, more preferably at least 90%, advantageously at least 95%, and more advantageously at least 98% homology).

The invention even further provides a method for preparing a food or feed supplement comprising a glucan polymer; the method comprising adding to the food or feed supplement an inventive glucanase enzyme or an inventive polypeptide having glucanase enzymatic activity, e.g., a polypeptide having a nucleotide sequence comprising SEQ ID NO: 1 or a fragment thereof such as a glucanase enzymatically active fragment of SEQ ID NO: 1 and/or polypeptide having homology to SEQ ID NO: 1 (with homology and fragment as herein discussed, e.g., at least 75%, preferably at least 85%, more preferably at least 90%, advantageously at least 95%, and more advantageously at least 98% homology and the polypeptide advantageously having glucanase enzymatic activity). The invention also provides a method for preparing a food or feed supplement comprising a glucan polymer; the method comprising adding to the food or feed supplement (i) a glucanase enzyme having the sequence shown as SEQ ID NO: 1 or (ii) a homologue or variant thereof having at least 75% homology thereto and the glucanase enzymatic activity of the enzyme shown as SEQ ID NO: 1 or (iii) a fragment of (i) or (ii) having the glucanase enzymatic activity. Advantageously the methods involve a glucanase enzyme having the sequence shown as SEQ ID NO: 1.

The invention also envisions a method for preparing a food or feed supplement comprising a glucan polymer; the method comprising adding to the food or feed supplement a glucanase enzyme, wherein the glucanase enzyme is coded for by a first nucleic acid molecule that hybridizes to a second nucleic acid molecule, and the second nucleic acid molecule has the nucleotide sequence as shown in SEQ ID NO: 2, and the glucanase enzyme has the glucanase enzymatic activity of the enzyme shown in SEQ ID NO: 1. The invention further encompasses a method for preparing a food or feed supplement comprising a glucan polymer; the method comprising adding to the food or feed supplement an inventive glucanase enzyme or glucanase enzymatically active fragment thereof and/or polypeptide having homology therewith, wherein the glucanase enzyme is coded for by a nucleic acid molecule having the nucleotide sequence as shown in SEQ ID NO: 2 or a nucleic acid molecule that is a fragment thereof or has homology thereto, e.g., with fragment and homology as herein discussed e.g., at least 75%, preferably at least 85%, more preferably at least 90%, advantageously at least 95%, and more advantageously at least 98% homology; advantageously, the gene product encoded by the nucleic acid molecule has glucanase enzymatic activity. These methods can also include determining the extent of glucan polymer degradation in the food or feed supplement. Also, the invention encompasses products from herein disclosed processes.

With respect to embodiments of the invention that comprise nucleotide or amino acid sequences, the invention can encompass fragments thereof, advantageously functional fragments thereof, as well as homologues thereof, advantageously functional homologues. A functional fragment or functional homologue advantageously has a function of the full length sequence. For instance, if the full length sequence is for an enzyme, advantageously the functional fragment or functional homologue has enzymatic activity. If the full length sequence encodes an enzyme, advantageously the functional fragment or functional homologue encodes a polypeptide that has enzymatic activity. If the full length sequence encodes a promoter, advantageously the functional fragment or functional homologue encodes that which also functions as a promoter. Thus, in addition to the disclosed sequences, the invention envisions sequences having at least 75%, preferably at least 85%, more preferably at least 90%, advantageously at least 95%, and more advantageously at least 98%, e.g., at least 99% or 100% homology to the disclosed sequences.

For instance, as to inventive nucleic acid molecules, the invention comprehends codon equivalent nucleic acid molecules. For instance, if the invention comprehends “X” protein having amino acid sequence “A” and nucleic acid molecule “N” encoding protein X, the invention comprehends nucleic acid molecules that also encode protein X via one or more different codons than in nucleic acid molecule N. Similarly, with respect to fragments or homologues of amino acid sequences, the invention envisions polypeptides wherein amino acids are substituted from those of disclosed sequences on the basis of charge and/or structural similarities. That is, in determining suitable fragments or homologues of disclosed amino acid sequences, the skilled artisan, without undue experimentation, can consider replacing amino acids in herein disclosed sequences with amino acids of similar charge and/or structure so as to obtain a fragment or homologue that is charge and/or structurally similar to herein disclosed sequences (in addition to or in the alternative to screening for activity such as enzymatic activity). Thus, the skilled artisan can consider charge and/or structure of herein disclosed sequences or portions thereof, in constructing fragments or homologues, without undue experimentation.

In addition, as to inventive nucleic acid molecules, the invention comprehends nucleic acid molecules that hybridize under stringent conditions as well as under high stringency conditions to herein disclosed nucleic acid molecules and, hybridizing or hybridization under stringent conditions and high stringency conditions can be synonymous with stringent hybridization conditions, terms which are well known in the art; see, for example, Sambrook, “Molecular Cloning, A Laboratory Manual” second ed., CSH Press, Cold Spring Harbor, 1989; “Nucleic Acid Hybridisation, A Practical Approach”, Hames and Higgins eds., IRL Press, Oxford, 1985; both incorporated herein by reference. Thus, specific hybridization of nucleic acid molecules according to the invention preferably occurs at stringent hybridization conditions. These nucleic acid molecules that specifically hybridize to herein disclosed nucleic acid sequences can be a probe or primer, for instance, for use in PCR for amplifying inventive DNA; and, these nucleic acid molecules can be any stretch of at least 8, preferably at least 10, more preferably at least 12, 13, 14, or 15, such as at least 20, e.g., at least 23 or 25, for instance at least 27 or 30 nucleotides in a herein defined nucleic acid molecule (sequence) which are unique thereto, e.g., not disclosed or suggested in prior art (e.g., not disclosed or suggested in documents such as Sakamoto et al., DDBJ Database, Accession No. D12901, Ooi et al., Swiss-prot 35 Database Accession No. p22669, Dalboge et al. geseq 32Database, Accession No. Q43452, Akiba, EP 0,458,162, and Okada, et al. Agric Biol Chem 49(5), 1257-65 that were cited and overcome in previous parent application U.S. application Ser. No. 08/913,264). As to PCR or hybridization primers or probes and optimal lengths therefor, reference is also made to Kajimura et al., GATA 7(4):71-79 (1990), incorporated herein by reference.

As to herein disclosed amino acid sequences, the invention comprehends nucleic acid molecules encoding the herein disclosed amino acid sequences, as well as novel nucleic acid molecules that hybridize under stringent conditions to nucleic acid molecules encoding herein disclosed amino acid sequences, as these nucleic acid molecules that hybridize under stringent conditions to nucleic acid molecules encoding herein disclosed amino acid sequences can provide proteins having similarity, homology or identity as herein discussed.

“Homology” is a well known term. Sequence homology or identity or similarity such as nucleotide sequence homology can be determined using the “Align” program of Myers and Miller, (“Optimal Alignments in Linear Space”, CABIOS 4, 11-17, 1988, incorporated herein by reference) and available at NCBI, as well as the same or other programs available via the Internet at sites thereon such as the NCBI site. Alternatively or additionally, the term “homology” or “identity”, for instance, with respect to a nucleotide or amino acid sequence, can indicate a quantitative measure of homology between two sequences. The percent sequence homology can be calculated as N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence similarity of 75% with the sequence AATCAATC (N_{ref}=8; N_{dif}=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur and Lipman, 1983 PNAS USA 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. Calif.). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences.

Additionally or alternatively, sequence identity or similarity or homology such as amino acid sequence similarity or identity or homology can be determined using the BlastP program (Altschul et al., Nucl. Acids Res. 25, 3389-3402, incorporated herein by reference) and available at NCBI, as well as the same or other programs available via the Internet at sites thereon such as the NCBI site. The following references (each incorporated herein by reference) provide algorithms for comparing the relative identity or homology or similarity of sequences such as amino acid residues of two proteins, and additionally or alternatively with respect to the foregoing, the teachings in these references can be used for determining percent homology or identity: Needleman S B and Wunsch C D, “A general method applicable to the search for similarities in the amino acid sequences of two proteins,” J. Mol. Biol. 48:444-453 (1970); Smith T F and Waterman M S, “Comparison of Bio-sequences,” Advances in Applied Mathematics 2:482-489 (1981); Smith T F, Waterman M S and Sadler J R, “Statistical characterization of nucleic acid sequence functional domains,” Nucleic Acids Res., 11:2205-2220 (1983); Feng D F and Dolittle R F, “Progressive sequence alignment as a prerequisite to correct phylogenetic trees,” J. of Molec. Evol., 25:351-360 (1987); Higgins D G and Sharp P M, “Fast and sensitive multiple sequence alignment on a microcomputer,” CABIOS, 5: 151-153 (1989); Thompson J D, Higgins D G and Gibson T J, “Cluster W: improving the sensitivity of progressive multiple sequence alignment through sequence weighing, positions-specific gap penalties and weight matrix choice, Nucleic Acid Res., 22:4673-480 (1994); and, Devereux J, Haeberlie P and Smithies O, “A comprehensive set of sequence analysis program for the VAX,” Nucl. Acids Res., 12: 387-395 (1984). And, without undue experimentation, the skilled artisan can consult with many other programs or references for determining percent homology.

As to determining glucanase enzymatic activity of polypeptides having such activity or that are glucanase enzymatically active polypeptides, such can be done from the teachings herein and the knowledge in the art without undue experimentation; for instance, note the testing of enzymatic activity set forth herein (and that the skilled artisan can employ other methods for determining enzymatic activity or can adapt other methods for determining enzymatic activity to the enzyme and substrate of the present invention, without any undue experimentation from the knowledge in the art and this disclosure). Likewise, as to determining enzymatic activity of a polypeptide in general, such can be done without undue experimentation from the knowledge in the art and the disclosure herein; for instance, by contacting the polypeptide with the substrate of the known enzyme under conditions under which the known enzyme would so function with respect to the substrate, and ascertaining whether the polypeptide indeed functions in a same or similar manner to the known enzyme with respect to the substrate. Similarly, one can screen for whether a fragment or homologue of a promoter is functional by inserting that fragment or homologue into a vector, upstream of a nucleic acid molecule encoding a protein of interest or a marker, and determining whether there is expression of that protein of interest or marker. Markers, such as antibiotics or resistance thereto, colored proteins or photo-emitting or luminescent proteins (e.g., tetracycline resistance, tetracycline, Beta-gal, luciferase, kanamycin, and the like) are known. Thus, determining whether a fragment or homologue of a promoter will function as a promoter can be done from the knowledge in the art, and this disclosure, without undue experimentation.

The present invention in at least one aspect is based on the identification and cloning of a novel nucleotide sequence encoding a novel endo-β-glucanase enzyme from the fungus Aspergillus niger. The novel endo-β-1,4-glucanase enzyme of the present invention, e.g., having the sequence set out in SEQ ID NO: 1 and/or encoded by SEQ ID NO: 2 (as well as fragments thereof or sequences homologous thereto as previously discussed) is highly advantageous; for instance, it can be used to prepare useful feeds containing cereals such as barley, maize and rice. Previously published documents have not disclosed or suggested either (i) the novel endo-β-1,4-glucanase enzyme(s) of the invention, e.g., having the sequence set out in SEQ ID NO: 1 and/or encoded by SEQ ID NO: 2 (as well as fragments thereof or sequences homologous thereto as previously discussed), or (ii) the uses of this novel enzyme in degrading glucans (very useful processes). Further, the present invention provides promoters and expression thereof with an inventive promoter.

Having thus described in detail preferred embodiments of the present invention, it is to be understood that the invention defined by the appended claims is not to be limited to particular details set forth in the above description as many apparent variations thereof are possible without departing from the spirit or scope of the present invention.

1. An enzyme obtainable from Aspergillus, wherein the enzyme has the following characteristics:
a. a MW of 24,235 D±50 D
b. a pI value of about 4
c. glucanase activity
wherein the glucanase activity is endo β-1,4-glucanase activity.

2. An enzyme having sequence shown as SEQ. I.D. No. 1 or a variant, homologue or fragment thereof.

3. An enzyme coded by the nucleotide sequence shown as SEQ. I.D. No. 2 or a variant, homologue or fragment thereof or a sequence complementary thereto.

4. A nucleotide sequence coding for the enzyme according to claim 1.

5. A nucleotide sequence coding for the enzyme according to claim 2.

6. A nucleotide sequence having the sequence shown as SEQ. I.D. No. 2 or a variant, homologue or fragment thereof or a sequence complementary thereto.

7. A nucleotide sequence according to any one of claims 4 to 6 operatively linked to a promoter.

8. A nucleotide sequence according to claim 7 wherein the promoter is the promoter having the sequence shown as SEQ. I.D. No. 3 or a variant, homologue or fragment thereof or a sequence complementary thereto.

9. A promoter having the sequence shown as SEQ. I.D. No. 3 or a variant, homologue or fragment thereof or a sequence complementary thereto.

10. A promoter according to claim 9 operatively linked to a GOI.

11. A promoter according to claim 10 wherein the promoter is operatively linked to a GOI, wherein the GOI comprises a nucleotide sequence according to any one of claims 4-6.

12. A terminator having the nucleotide sequence shown as SEQ. I.D. No. 13 or a variant, homologue or fragment thereof or a sequence complementary thereto.

13. A signal sequence having the nucleotide sequence shown as SEQ. I.D. No. 14 or a variant, homologue or fragment thereof or a sequence complementary thereto.

14. A construct comprising or expressing the invention according to any one of claims 1 to 13.

15. A vector comprising or expressing the invention of any one of claims 1 to 14.

16. A plasmid comprising or expressing the invention of any one of claims 1 to 15.

17. A transgenic organism comprising or expressing the invention according to any one of claims 1 to 16.

18. A transgenic organism according to claim 17 wherein the organism is a fungus.

19. A transgenic organism according to claim 17 wherein the organism is a filamentous fungus, preferably Aspergillus.

20. A transgenic organism according to claim 17 wherein the organism is a plant.

21. A transgenic organism according to claim 17 wherein the organism is a yeast.

22. A process of preparing an enzyme according to any one of claims 1 to 3 comprising expressing a nucleotide sequence according to any one of claims 4-8.

23. A process according to claim 22 wherein the enzyme has the sequence shown as SEQ. I.D. No. 1 or a variant, homologue or fragment thereof, and the nucleotide sequence has the sequence shown as SEQ. I.D. No. 2 or a variant, homologue or fragment thereof or a sequence complementary thereto.

24. A process according to claim 22 or claim 23 wherein the expression is controlled (partially or completely) by use of a promoter according to claim 9.

25. A process for expressing a GOI by use of a promoter, wherein the promoter is the promoter according to claim 9.

26. Use of an enzyme according to any one of claims 1 to 3 or prepared by a process according to any one of claims 22 to 25 to degrade a glucan.

27. Plasmid NCIMB 40704, or a nucleotide sequence obtainable therefrom for expressing a glucanase enzyme or for controlling the expression thereof or for controlling the expression of another GOI.

28. A glucanase enzyme having the ability to degrade β-1,4-glucosidic bonds, which is immunologically reactive with an antibody raised against a purified glucanase enzyme having the sequence shown as SEQ. I.D. No. 1.

29. A signal sequence having the sequence shown as SEQ. I.D No. 15 or a variant, homologue or fragment thereof.
